(19) United States
(12) Patent Application Publication
(10) Pub. No.: US 2025/0222179 A1
(43) Pub. Date: Jul. 10, 2025

(54) DEVICES OF RESORBABLE DRUG-ELUTING SHAPE MEMORY FOAM

(71) Applicants: Baylor College of Medicine, Houston, TX (US); Board of Regents, The University of Texas System, Austin, TX (US); The Texas A&M University System, College Station, TX (US)

(72) Inventors: Julie Hakim, Houston, TX (US); Elizabeth Cosgriff-Hernandez, Austin, TX (US); Melissa Grunlan, College Station, TX (US)

(73) Assignees: Baylor College of Medicine, Houston, TX (US); Board of Regents, The University of Texas System, Austin, TX (US); The Texas A&M University System, College Station, TX (US)

(21) Appl. No.: 18/853,916

(22) PCT Filed: Apr. 4, 2023
(86) PCT No.: PCT/US2023/017465

§ 371 Date: Oct. 3, 2024

Related U.S. Application Data
(60) Provisional application No. 63/326,996, filed on Apr. 4, 2022.

(51) Int. Cl. A61L 31/14; A61F 6/08; A61L 31/06

(52) U.S. Cl. CPC A61L 31/146; A61F 6/08; A61L 31/06; A61L 31/148; A61L 2400/16

(57) ABSTRACT

Embodiments of the disclosure concern devices comprised of resorbable, shape-memory foam configured to allow for healing at a desired location. In particular embodiments, use of the device at the location utilizes the inherent environment at the location to allow expansion of the device, resulting in an improved fit for the device. In specific cases, the device is a vaginal stent configured for use for pediatric or adolescent populations.

This application claims priority to U.S. Provisional Patent Application Ser. No. 63/326,996, filed Apr. 4, 2022, which is incorporated by reference herein in its entirety.

This invention was made with government support under HD104059 awarded by the National Institutes of Health. The government has certain rights in the invention.

### I. TECHNICAL FIELD

This disclosure relates at least to the fields of chemistry, devices, wound care, and medicine.

### II. BACKGROUND

Many conditions such as post-radiation fibrosis, urinary incontinence, vaginismus, and pelvic organ prolapse can cause vaginal scarring and subsequent closure that may require vaginal reconstruction surgery. Currently, more than 8.9 million girls and women fall into this patient population who experience immense pain and suffering. Pediatric and adolescent gynecologic surgeons often use vaginal stents in the immediate postoperative and post-radiation settings to facilitate wound healing. Unfortunately, the only available vaginal stents on the market are large, non-ergonomic designs that are poorly tolerated in the pediatric population. Indeed, these stents only recently become available again after being removed from production for more than 5 years. As a result, many surgeons have resorted to make-shift devices created in the operating room. All of these present deployment challenges, limited sizing options, and poor retention, which frequently requires suturing of the vulvar lips to prevent egress of the stent. There is a critical need for a new pediatric/adolescent-specific vaginal stent with appropriate sizing that can apply constant pressure to the boundaries of the neovagina (reconstructed or newly constructed vagina) to prevent fibrosis and maintain vaginal caliber during wound healing. A degradable stent would serve as a more individualized and comfortable alternative to both treating and preventing vaginal scarring.

### SUMMARY OF THE DISCLOSURE

Embodiments of the disclosure include compositions and methods for tissue care of any kind, including treatment for a wound, diseased tissue, or medical condition of any kind that impacts a location in vivo. In specific embodiments, the compositions and methods concern medical devices for treatment for a wound, diseased tissue, or medical condition of any kind. Although the device can be utilized for anyone that is in need, in specific embodiments the device is for a mammal, including a human, and the mammal may comprise a urinary tract, vagina, fallopian tube, ovary, cervix, uterus, bladder, ureter, urethra, vas deferens, epididymis, testicle, or seminal vesicle in need of the device. In specific embodiments, the individual in need is an individual having a vagina and/or cervix that is wounded, diseased, in need of post-operational treatment, in need of gender reassignment, and/or in which a medical condition has resulted in need of treatment of the vagina or cervix. A wound may be from damage during sexual intercourse, use of a foreign object, scarring, shortening, or tightening of the tissue from surgery and/or radiation, injury in the crotch area (such as straddle injury), menopause, and so forth. Post-operational treatment may require use of the device, such as from obstetric surgery or procedure, vaginoplasty, labiaplasty, male-to-female transgender surgery, and so forth. In some cases, the device is utilized for women following pelvic radiation, including those that suffer from fibrosis, such as high levels of fibrosis. The device may be used for reconstructive or cosmetic purposes (e.g. vaginal rejuvenation), in some cases. The device may be used as a degradable contraceptive device, such as an intrauterine device (IUD) in a form of birth control or a contraceptive implant of any kind. The device may be used as a stent for any tissue in need thereof, biological male or biological female. In specific embodiments, the device is used as a stent for any diseased or damaged part of a biological male or biological female reproductive tract including fallopian tube(s), vagina, uterus, ovary, cervix, vas deferens, epididymis, testicle, rectum, or seminal vesicle. In specific embodiments, the device is used in an individual with pelvic inflammatory disease, an ectopic pregnancy, incontinence for any reason, as a vaginal suppository (e.g., for pathogen treatment, such as bacteria, virus, or fungus), for genito-urinary symptoms associated with menopause, and so forth.

The device may comprise a particular material, including non-metal material, and/or a particular shape. The device may be obtained off-the-shelf or may be manufactured upon need of the device. In either case, the type of need and/or size of the individual receiving the device may be taken into account prior to manufacture or selection of the device. In certain embodiments, the individual receiving the device is pediatric (up to 12 years of age) or adolescent (12-18 yrs of age), although in some cases the device may be configured for an adult. The device may be designed or configured to be utilized for temporary use, such as being made of a material that disappears or dissolves over time, including being resorbed by the body.

In specific embodiments, the device has certain design features, such as at least those that would include easy insertion with improved ergonomics, prevention of egress with Valsalva, prevention or treatment of fibrosis, prevention or treatment of stenosis, improved vaginal or cervical healing or prevention of cervical dilation, as a contraceptive, as a stent at least for fallopian tube(s), vagina, ovary, cervix, vas deferens, epididymis, testicle, or seminal vesicles, with pelvic inflammatory disease, an ectopic pregnancy, incontinence for any reason, as a vaginal suppository (e.g., for pathogen treatment, such as bacteria, virus, or fungus), for genito-urinary symptoms associated with menopause, and so forth. for example. In specific cases, the device is configured to be placed in tissue, including a canal such as a vagina to aid in healing. In specific embodiments, the device is a tubular support placed temporarily in the vagina. In particular embodiments, the device is biocompatible and may be resorbable. In cases wherein the device is a resorbable stent, its development provides additional advantages by eliminating the need for postoperative stent removal after wear-time is complete. In the design of a resorbable stent, in at least some cases one balances mechanical properties such that the stent maintains tissue patency (e.g., sufficient radial strength) while avoiding adverse biological responses, such as tissue overgrowth, and ensuring sufficient flexibility for patient comfort. The device may elute one or more contraceptives and/or one or more biological agents, such as therapeutic compositions of any kind, including those that enhance wound healing, prevent or reduce the risk of infection, treat pain, treat or reduce the risk of scarring and/or fibrosis, and so forth.

In particular embodiments, the device is a resorbable, shape-memory vaginal stent that can improve clinical outcomes and quality of life for pediatric and adolescent patients, as well as adult women, such as following vaginal surgery, radiation, or any of the vaginal injuries/disease states referred to elsewhere herein. In specific embodiments, the design may be used to retain or restrict pathologic physiologic processes such as preventing fibrosis or pre-term birth, such as because of cervical incompetence or vaginal/uterine prolapse. In specific embodiments, the design utilizes a shape-memory foam that is biodegradable and can assume a secondary, compressed shape for ease of deployment. Upon insertion, the change in temperature and/or hydration (for example) initiates the expansion of the foam to shape fit to the individual patient and in specific embodiments also restores the lumen of the stent to allow egress of vaginal secretions. Uniquely, this circumvents the need for an external balloon to inflate the stent, a strategy common to current designs that causes challenges during clinical deployment. The mechanical properties, shape recovery (i.e., expansion), and resorption kinetics may be precisely controlled, such as by a combination of polymer chemistry and pore size control.

In one embodiment, there is a vaginal or cervical device, comprising a self-fitting, resorbable shape memory foam. In specific embodiments, the foam is comprised of or derived from poly(vinyl alcohol) (PVA), poly-L-lactic acid (PLLA), polyglycolic acid (PGA), polyhydroxyethyl-methacrylate (PHEMA), poly(N-isopropylacrylamide) (PNIPAAm), [2-(methacryloyloxy)ethyl]dimethyl-(3-sulfopropyl)ammonium hydroxide (MEDSAH), 3-(acrylamidopropy)trimethylammonium chloride (AAPTAC), poly(ethylene glycol) diacrylate (PEG-DA), poly(tetrafluoroethylene) (PTFE), Poly(ε-caprolactone) (PCL), polycaprolactone diacrylate (PCLDA), polyvinylpyrrolidone (PVP), diacrylated poly(ethylene glycol) (PEG-DA), poly(2-acrylamide-2-methyl-propane sulfonic acid) (PAMPS), poly([2-(methacryloyloxy)ethyl]dimethyl(3-sulfopropyl)ammonium hydroxide) (PMEDSAH), polyurethane, polyether-ether-ketone, polydimethylsiloxane (PDMS), or any combination or copolymer thereof. The device may be of any shape, including a cylinder, a ring, a cube, a disc, or it may be bowl-shaped or saddle-shaped. The device may be part of a vaginal patch or graft.

In various embodiments, the size of the device may be expandable upon placement in vivo. In specific cases wherein the device is in the shape of a cylinder, the length of the cylinder prior to placement in vivo may be in the range of about 0.5 inch to about 2 inches. In specific cases wherein the device is in the shape of a cylinder, the radius of the lumen may be about 0.1 inch to about 0.25 inch. In specific embodiments, the device is in the shape of a cylinder, and the width of the wall of the cylinder may be about 0.05 inch to about 0.25 inch. In any embodiment of the disclosure, the size of the device (including any stent for any purpose), may be tailored or selected to the appropriate tissue in need. For example, the device being used in the vagina would have a diameter larger than a device being used in a fallopian tube (on the order of millimeters).

In particular embodiments, the device is a vaginal stent. The size of the device may be configured for a vagina of a pediatric, adolescent, or adult individual. In specific embodiments, the device is a vaginal ring or cervical ring. The device may be further defined as comprising multiple devices of concentric cylindrical shapes. The device may comprise an effective amount of one or more agents, and in some cases the one or more agents elutes from the device, is a coating on the device, or both. The agent may be for contraception, wound healing and/or scar prevention. In specific embodiments, the agent is a hormone, antibiotic, pain reliever, hemorrheologic, vasoconstrictive, anti-inflammatory, anti-fibrotic, wound-healing agent, radioprotective material, anti-fungal, anti-viral, contraceptive, spermicide, or any combination thereof. The agent may comprise AMD3100, tacrolimus, 2-octyl cyanoacrylate, Alevicyn, Artiss, Becaplermin, Betaine/polyhexanide, Cadexomer iodine. Collagenase, Dermabond, Eletone cream, Episalvan, Evicel, Fibrin sealant, Filsuvez, Hypochlorous acid topical, Lodosorb, NexoBrid, Oleogel-S10, Petrolatum & mineral oil topical, Prontosan, Proteolytic enzyme, Regranex gel, Santyl, TachoSil, Tisseel VH, Tropazone, progestin, etonogestrel, or a combination thereof.

Embodiments of the disclosure include methods of treating a wound or diseased tissue or medical condition, comprising the step of applying any device encompassed herein to the wound or diseased tissue. The wound may be in the vagina or cervix. The wound may be from a medical procedure, including an obstetrical procedure. In specific embodiments, the wound or diseased tissue or medical condition comprises treatment after radiation and/or surgery, fibrosis, post-operational treatment after gender reassignment, physical damage or injury, scarring for any reason, shortening and/or tightening of tissue from surgery and/or radiation, menopause, vaginal birth, post-operational treatment after vaginal birth, vaginoplasty, labiaplasty, prevention of pre-term birth, cervical incompetence, vaginal/uterine prolapse, bacterial vaginosis, yeast infection, lichen planus, lichen sclerosus, incontinence, or a combination thereof. In specific embodiments, the device is in the shape of a ring and is applied to the vagina or cervix.

In certain embodiments, there is a method of treating a wound or medical condition at a desired location in vivo, comprising the step of placing any device encompassed herein to the desired location, wherein following placement at the location, the device expands in size thereby improving fitting at the desired location.

In some embodiments, there is a method of manufacturing any device encompassed herein, comprising the steps of: (a) forming a biocompatible and resorbable three-dimensional device by one or more of the methods of: emulsion templating, salt leaching, gas-foaming, electro-spinning, and 3D-printing, wherein the three-dimensional device is comprised of or derived from a polymer selected from the group consisting of poly(vinyl alcohol) (PVA), poly-L-lactic acid (PLLA), polyglycolic acid (PGA), polyhydroxyethyl-methacrylate (PHEMA), poly(N-isopropylacrylamide) (PNIPAAm), [2-(methacryloyloxy)ethyl]dimethyl-(3-sulfopropyl)ammonium hydroxide (MEDSAH), 3-(acrylamidopropy)trimethylammonium chloride (AAPTAC), poly(ethylene glycol) diacrylate (PEG-DA), poly(tetrafluoroethylene) (PTFE), Poly(ε-caprolactone) (PCL), polycaprolactone diacrylate (PCLDA), polyvinylpyrrolidone (PVP), diacrylated poly(ethylene glycol) (PEG-DA), poly(2-acrylamide-2-methyl-propane sulfonic acid) (PAMPS), poly([2-(methacryloyloxy)ethyl]dimethyl(3-sulfopropyl)ammonium hydroxide) (PMEDSAH), polyurethane, polyether-ether-ketone, polydimethylsiloxane (PDMS), and a combination thereof; and, optionally, (b) either during or after production of said three-dimensional device, providing at least one therapeutic agent within and/or on at least one surface of the three-dimensional device. The therapeutic agent may be a hormone, antibiotic, pain reliever, hemorrheologic, vasoconstrictive, anti-inflammatory, anti-fibrotic, wound-healing agent, radioprotective material, or any combination thereof. The device may be placed at a desired location in vivo in an individual.

Embodiments of the disclosure provide a device that comprises, consists of, or consists essentially of resorbable, shape-memory material, including resorbable, shape-memory foam. The device may or may not be of a particular shape, may or may not be of a particular shape-memory material, and may or may not be of a particular size. The device may have one or more apertures, and when multiple apertures are utilized, they may or may not be of the same size. The dimension(s) of the device may or may not be tailored for a particular application, including for a particular-sized recipient. In specific embodiments, the device is for use off-the-shelf. In some embodiments, the device is manipulated prior to use, such as manipulations with respect to its size (including at least diameter, width, height, etc.), material, shape, the presence of one or more apertures, and so forth. In some embodiments, the device may be manipulated to comprise one or more agents, which may or may not elute from the device; the agent may be provided as a coating on the device, in specific cases. In specific embodiments, the agent is for contraception, wound healing, scar prevention, and/or pathogen treatment. The agent may or may not be one or more hormones, antibiotics, pain relievers, hemorrheologics, vasoconstrictives, anti-inflammatory(ies), anti-fibrotics, wound-healing agents, radioprotective materials, anti-fungals, anti-virals, contraceptives, or any combination thereof.

In particular embodiments, the device and methods of the disclosure are utilized for individuals following radiation. In such cases, the individual is provided the device before, during, and/or after exposure of at least one tissue of the individual to radiation of any kind. In specific cases, the device comprises an agent that treats or prevents burning of the tissue from the radiation.

In some embodiments, the disclosure concerns methods and treatments for maintaining an opening of a tissue, or preventing collapse of a tissue (including in some cases an opening in a tissue), or both. In specific examples, the device is utilized in a fallopian tube following an ectopic pregnancy to maintain the opening of the fallopian tube and/or to provide therapy at the site of the damaged tissue of the fallopian tube. In another example, the device is used for an individual that has or is at risk of having pelvic inflammatory disorder (PID). In another example, the individual in need of the device is incontinent for any reason or has one or more genito-urinary symptoms associated with menopause. The device may be used in a biological female following childbirth, miscarriage, abortion, or onset of menopause. The device may be used in an individual that has a sexually transmitted disease or is at risk of same, including syphilis, gonorrhea, chlamydia, HIV, Human Papillomavirus, Herpes, or a combination thereof.

In particular embodiments, the disclosure concerns methods and devices for use in reproduction, either to enhance contraception and viability of the embryo in utero or to reduce the risk of pregnancy. In specific cases, the device is utilized as a degradable intra-uterine device or as a contraceptive implant. In contraceptive embodiments, the device may be manipulated to comprise one or more agents for contraception prior to its in vivo use.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The disclosure may be better understood by reference to one or more of these drawings in combination with the detailed description presented herein.

FIG. 1. One embodiment of SMP foam stent with conformable expansion.

FIG. 2. Examples of innovative features of the resorbable, shape-memory foam stent.

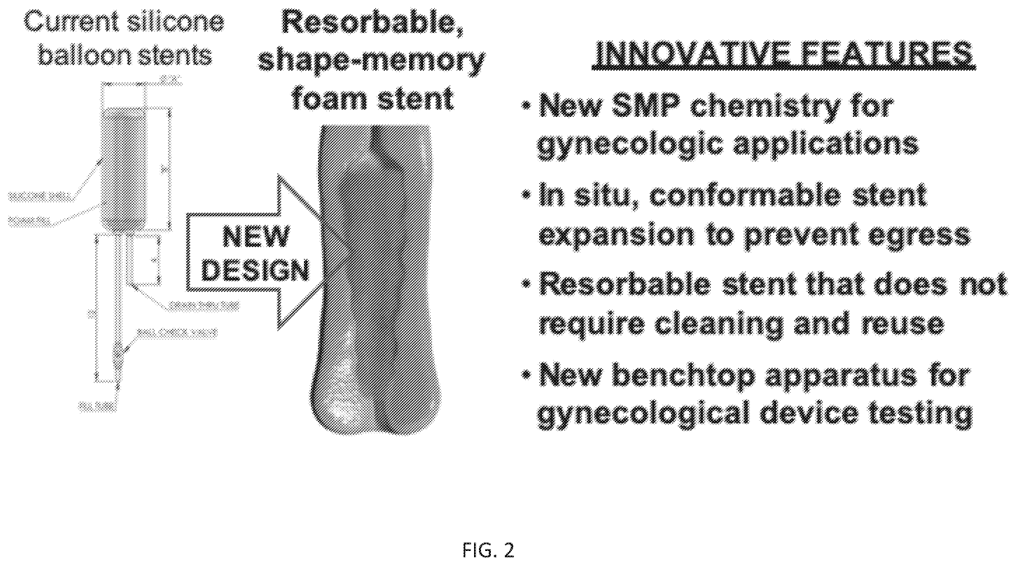

FIG. 3. PCL-based SMP foam stent with demonstrated shape recovery.

FIG. 4. Mechanical testing of SMP foams using custom setup to approximate Valsalva.

FIG. 5. 3D-printed pelvic organs in acrylic box (Lazarus3D™.)

FIG. 6. One example of a workflow schematic of in vivo studies.

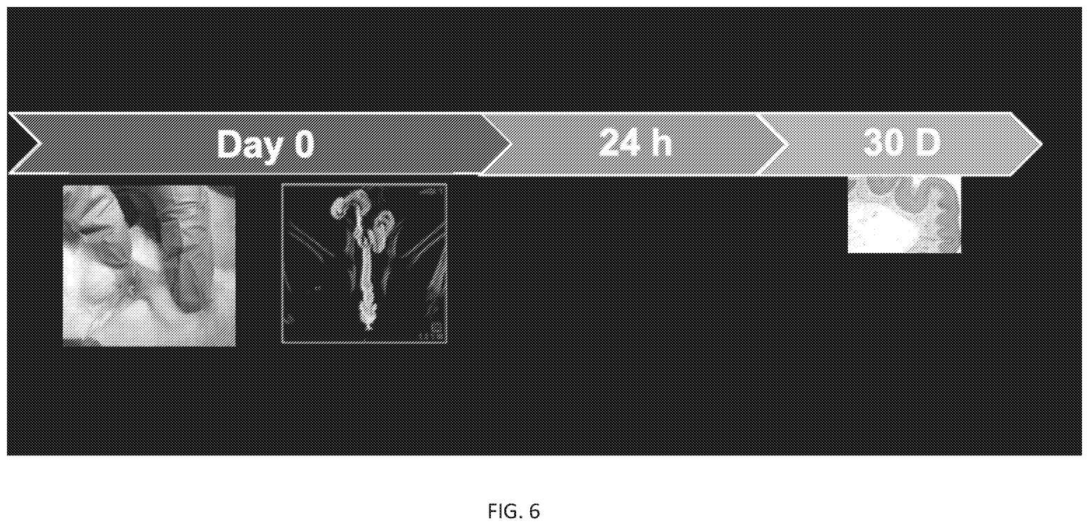

FIGS. 7A-7E. (FIG. 7A) Fabricated PCLDA foam; FIG. 7B) Micrograph of foam structure; (FIG. 7C) Shape memory recovery from compressed state; (FIG. 7D) Time to recovery at a given temperature; (FIG. 7E) Stent insertion in rabbit vagina.

FIGS. 8A and 8B. FIG. 8A. Impact of altering polymer architecture on various other polymer properties. FIG. 8B. The six compositions to be characterized.

FIGS. 9A-9C. FIG. 9A Thermograph of a representative DSC run. FIG. 9B Tₘ and FIG. 9C crystallinity of scaffold compositions *p<0.5 compared to respective control. #p<0.5 between M,,.

FIGS. 10A-10C. Compressive (FIG. 10A) modulus (FIG. 10B) strength and (FIG. 10C) toughness of porous scaffolds.

FIGS. 11-11C. Mass loss of linear- and star-PCL scaffolds at (FIG. 11A) 10k gmol⁻¹, (FIG. 111B) 7.5k gmol⁻¹, and (FIG. 11C) 5k gmol-1

FIGS. 12A-12C. FIG. 12A. SEM images of the scaffold cross-sections. FIG. 12B. Porosity between all compositions was maintained between 60-80%. FIG. 12C. Scaffold pore sizes #p>0.05 linear vs star.

FIG. 13. Pore interconnectivity based on a qualitative wicking test

FIGS. 14A and 14B. TGA of solid films to verify crosslinking within the films and to analyze thermal degradation rates for (FIG. 14A linear-PCL-DA compositions and (FIG. 14B) star-PCL-TA compositions.

FIGS. 15A and 15B. Qualitative crosslink density. (FIG. 15A) images of a dry and swollen solid film for each composition. (FIG. 15B) diameter of the dry and swollen samples were taken via calipers and calculated to get the percentage of change in the diameter.

FIG. 16 provides an example of demonstration of SMP vaginal stent design and deployment.

FIG. 17 shows the effect of PCL-DA:Toluene concentration and NVP on emulsion templated foam structure.

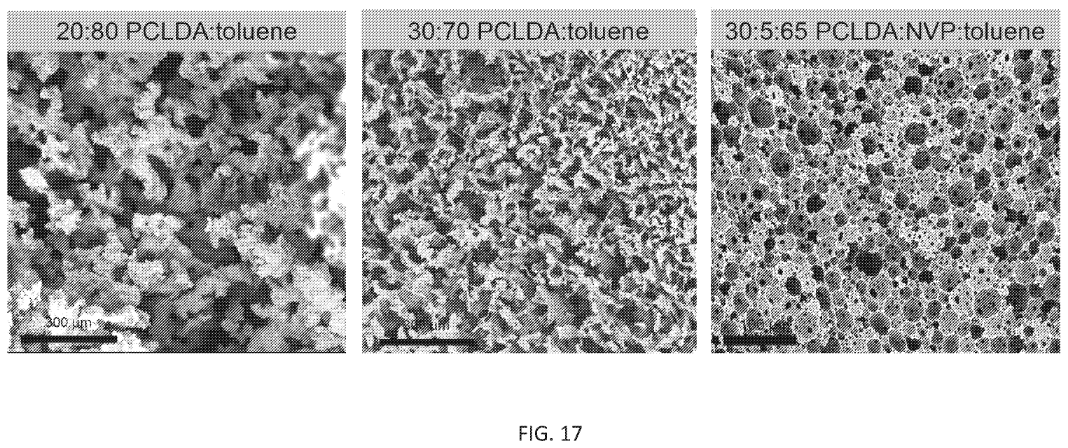

FIG. 18 shows the effect of macromer molecular weight on the transition temperature (Tₘ) of star-PCL SMP.

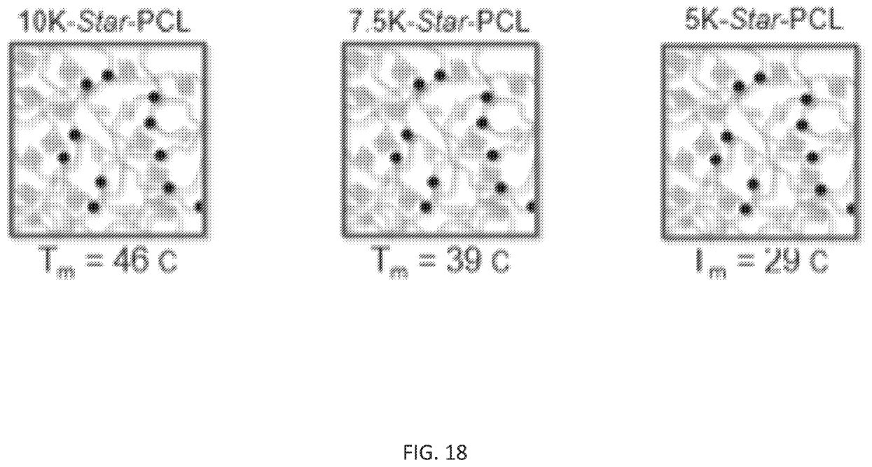

### DETAILED DESCRIPTION

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa, and different embodiments may be combined. Furthermore, compositions of the disclosure can be used to achieve methods of the disclosure. The claims originally filed are contemplated to cover claims that are multiply dependent on any filed claim or combination of filed claims.

### I. Examples of Definitions

As used herein, the term “resorbable” refers to the capacity of a device to undergo biodegradation (chemical breakdown by biological agent) and the degradation products removed by cellular activity in a biological environment.

As used herein, the term “shape fit” or “shape fitting” or “self fit” or “self fitting” refers to the expansion of the device to conform to the inner wall of a vagina, including a vagina in need of repair or newly constructed or reconstructed, such that at least in some cases the device applies constant pressure to at least part of the vagina wall.

As used herein, the term “memory foam” refers to a shaped, porous material that can be deformed and can return to its original, predeformed shape after being deformed.

### II. General Embodiments

Women have historically been excluded at all levels of biomedical research and medical device innovation that has led to a gaping hole across the global healthcare landscape as it pertains to meeting even the most basic needs in women's health. For issues specifically pertaining to vaginal health of young girls, the unmet needs are startling. As many as 50,000 patients per year in North America require surgical reconstruction of a new vaginal canal (Emans et al., 2012). Recent increases in malformations such as specific congenital genitourinary anomalies (Lloyd et al., 2013) and a societal shift towards better acceptance of male-to female transgender surgeries have led to a surge in the number of surgeries for neo-vaginal creation in girls and young women. The success of this procedure depends upon preventing the apposition of the vaginal tissues during the post-surgical period (Emans et al., 2012), with loss of patency of the vaginal canal during healing leading to severe postoperative complications. Most commonly, patients experience restenosis and scar tissue formation, which can occur in up to 73% of patients, necessitating persistent perineal dilatation using vaginal dilators after surgery in order to prevent tissue apposition and maintain neo-vagina caliber and length (Raya-Rivera et al., 2014). The only method to maintain the vaginal caliber is with the use of vaginal dilators; however, this approach fails to be patient-forward by requiring substantial patient effort with frequent and painful insertion in prone positions that interferes with daily activities. As a result, adherence to the recommended dilation regimes is low, with less than 50% using dilators at the recommended frequency, and as many as 75% stopping the use of the dilator within a year (Law et al., 2015).

Noncompliance or misuse of vaginal dilators is associated with significant complications including vaginal bleeding or even perforation of the vagina or bladder and fistula formation (Patel et al., 2016). Many pediatric surgical centers quote a risk of surgical revision after vaginal reconstruction to be ˜50%. Prevention of these complications would offer substantial benefits to both immediate and long-term quality of life of this underserved population.

Despite being one of the only ways to prevent the need for perineal dilation and subsequent surgical revision, adult vaginal stents have only recently become available again after having been removed from production for more than 5 years. Post-operative difficulties are even more problematic in the pediatric population given that no commercially available vaginal stent has ever existed for adolescents. Although prosthodontists can create customized vaginal stents, they are expensive, not widely available, and the fabricated design requires anticipation of the eventual surgically corrected size of the vagina, which could lead to ill-fitting stents and all of the sequalae that this entails. Because of the non-existence of a commercially available product and little availability/poor results of prosthodontist-designed stents, physicians are relegated to creating their own makeshift stents for their gynecology patients, using finger slots from sterile gloves and gauze, sterile cement coated in bone wax and placed in a condom, or plastic molding. All of these options present deployment challenges, limited sizing options, and poor retention, which frequently requires suturing of the vagina closed to prevent egress. In such cases, the labia is stitched together to immobilize the vaginal dilator in place, causing pain and suffering at a minimum and an elevated risk of menstrual build-up and infection. Over the course of time that a patient's labia are sutured, these girls also require an indwelling bladder catheter, increasing hospitalization time and morbidity. The need for vaginal stents appropriately designed for this patient population cannot be overstated.

The disclosure provides devices and methods that may be utilized in post-operative settings, post-radiation settings, for contraceptive purposes, for prevention purposes, for therapeutic purposes, etc.

### III. Device and Uses Thereof

The present disclosure concerns devices using memory shape material that may be utilized for gynecological and/or urological purposes, for example. In particular embodiments, the device is resorbable, self-fitting upon placement, or both. In certain embodiments, the memory shape material from which the device is made allows for the self-fitting of the device into the body at a desired location. In particular embodiments, the device expands to become self-fitting following its delivery in vivo. In specific embodiments, the device is suitable for placement in the urinary tract, vagina, fallopian tube, ovary, cervix, uterus, bladder, ureter, urethra, vas deferens, epididymis, testicle, rectum, or seminal vesicle, as examples. The shape of the device may also be particular and specifically designed/utilized such that it provides optimum function in the desired location of the body. For example, in specific cases the device is a stent, and the shape of the stent may be such that normal processes are allowed to occur in the individual in spite of its presence. For example, the shape and design of the device may be such that body fluids from the site of placement are free to pass from the body. In a specific embodiment, the device is a vaginal stent, and the shape and design of the stent may be such that vaginal discharge of any kind may pass through the stent and outside of the body of the individual. In such cases, the device will include an opening through it, and in specific embodiments the vaginal stent is generally cylindrical.

Specific embodiments encompass the design of a resorbable, self-fitting vaginal stent that can improve clinical outcomes and quality of life for any individual, including pediatric and adolescent patients, such as those following a vaginal procedure or medical care of any kind, including surgery. In particular embodiments, the vaginal stent will be configured to allow ease of deployment (‘self-expanding’), prevention of egress with Valsalva, and maintenance of vaginal caliber. In specific embodiments, prevention of tissue apposition during healing ultimately prevents deleterious conditions, such as fibrosis. In addition, the development of a single-use, resorbable stent provides additional advantages by eliminating the need for post-operative stent removal after wear-time is complete.

In particular embodiments, the device is configured for use in a temporary manner or for long-term care. In specific embodiments, the device is configured for use for about 3-12 months. Specific embodiments allow for the material of the device to be resorbable such that over the course of its use, the material is resorbed by adjacent tissue until the device is completely resorbed. In alternative embodiments, the material is resorbed by adjacent tissue during the course of its use, yet the remainder of the device is removed after a suitable period of time. The resorption of the device may or may not conform to the timing of the healing. For example, the tissue may be healed prior to complete resorption of the device, or the tissue may not be healed completely prior to complete resorption of the device, and an additional device may then be utilized if needed.

The device may be configured as a particular shape for the purpose of optimal function following placement at a desired location, such as the vagina or cervix. The shape may be of any kind that suits its purpose, but in specific embodiments the device is a cylinder, such as for healing in a vagina, or as a ring, such as for vaginal/cervical use, including as part of a contraceptive device, for example. Examples of shapes for the device include cylindrical, circular (such as a ring), capsule (cylindrical with hemispherical ends), planar sheets, disc, or other shapes. The device can be manipulated by a healthcare provider to have a desired shape and/or dimensions. In particular embodiments, the device comprises one or more apertures for which fluid may pass through following placement in vivo.

In particular uses for the vagina, the device may be cylindrical, and in cases where the device is cylindrical, the radius of its lumen, width of its wall, and/or length of the cylindrical shape of the device may be of any sufficient size to allow the device to function appropriately. For example, in cases wherein the device is a vaginal stent, the thickness of the wall of the cylinder, the length of the cylinder, and the diameter of the opening of the cylinder may be of certain size ranges, including both prior to placement in vivo and upon placement in vivo. As one example, the length of the cylinder prior to placement in vivo is in the range of about 0.5 inch to about 2 inches. In another example, the radius of the lumen prior to placement in vivo is about 0.1 inch to about 0.25. In another example, the width of the wall of the cylinder prior to placement in vivo is about 0.05 inch to about 0.25 inch. In certain cases, the stent has appropriate sizing that can apply constant pressure to the vaginal wall, and such pressure prevents or reduces the likelihood or severity of fibrosis and maintains vaginal caliber during wound healing. In a specific case, the stent applies constant pressure to the boundaries of a neovagina to prevent or reduce the likelihood or severity of fibrosis to improve healing.

Specific examples of uses of the device include those for postoperative and post radiation settings, such as to facilitate wound healing or prevent scarring or occlusion. In any event, particular examples include at least treatment after radiation and/or surgery, fibrosis of any kind or degree and including treatment or prevention, post-operational treatment after gender reassignment, physical damage or injury (such as from sexual intercourse or use of a foreign object), scarring for any reason, shortening and/or tightening of tissue from surgery and/or radiation, menopause, vaginal birth, post-operational treatment after vaginal birth, vaginoplasty, labiaplasty, prevention of pre-term birth, cervical incompetence, vaginal/uterine prolapse, bacterial vaginosis, yeast infection, lichen planus, lichen sclerosus, incontinence, or a combination thereof. In these cases the device may or may not be cylindrical. For example, for prevention or delay of pre-term birth, the device may be ring-shaped.

In some cases, one or more degradable devices are encompassed within part or all of another degradable device, thereby allowing for continuous expansion of the vagina and/or continuous delivery of a bioactive agent.

Embodiments of the disclosure include methods of treating a tissue in an individual, comprising the step of applying any device encompassed herein to tissue of the individual. The tissue may be wounded tissue, diseased tissue, or where the individual has a medical condition associated with tissue in need of the device. In specific cases, the wound or diseased tissue is in the urinary tract, vagina, fallopian tube, ovary, cervix, uterus, bladder, ureter, urethra, vas deferens, epididymis, testicle, rectum, or seminal vesicle. The tissue may or may not be from wound from a medical procedure, such as an obstetrical procedure, including at least childbirth, miscarriage, abortion, radiation, cancer treatment, biopsy, and so forth. In specific embodiments, the wound or diseased tissue or medical condition comprises treatment after radiation and/or surgery, for fibrosis, post-operational treatment after gender reassignment, physical damage or injury, scarring for any reason, shortening and/or tightening of tissue from surgery and/or radiation, menopause, vaginal birth, post-operational treatment after vaginal birth, vaginoplasty, labiaplasty, prevention of pre-term birth, cervical incompetence, vaginal/uterine prolapse, bacterial vaginosis, yeast infection, lichen planus, lichen sclerosus, incontinence, pelvic inflammatory disease, ectopic pregnancy, genito-urinary symptoms associated with menopause, or a combination thereof.

Embodiments of the disclosure include methods of maintaining an opening and/or preventing collapse of one or more tissues of an individual, comprising the step of placing in the tissue any device encompassed herein. The tissue may be in the urinary tract, vagina, fallopian tube, ovary, cervix, uterus, bladder, ureter, urethra, vas deferens, epididymis, testicle, rectum, or seminal vesicle. In some cases, the device comprises one or more therapeutic or contraceptive agents.

In one particular embodiment, the disclosure provides fabrication of resorbable, shape-memory foams that provide an improved, conformable fit. Poor retention and need for secondary procedures for removal are the primary clinical challenges of current vaginal stents. In specific embodiments, there is adaptation of poly(e-caprolactone)-based shape memory foams for use in devices, including at least vaginal stents, to provide conformable fits that will improve patient comfort and stent retention. One can tune the transition temperature (for expansion) and pore architecture of the foams to achieve the clinically-relevant deployment parameters (shape expansion, expansion time, mechanical properties and resorption rate). In some cases, one can use a custom anatomical benchtop model that replicates the anatomy and forces in the vagina to screen candidate stents and ensure adequate deployment and retention prior to testing in vivo. Mechanical testing as a function of degradation may be used to ensure that the stent retains sufficient mechanical properties to maintain vaginal caliber over a particular target healing time, such as 4-6 weeks.

In one particular embodiment, the disclosure provides for assessment of resorbable foams in a rabbit model to confirm in vivo deployment and retention. Rabbits are frequently used as a gynecologic model and can provide complementary information regarding deployment, retention, and tolerance of devices of the disclosure, including vaginal stents. To this end, one can adapt the stent composition identified in embodiments described elsewhere herein to sizes appropriate for testing in the rabbit model. Acute testing assesses the ease of deployment, shape fit with imaging, and retention in the vagina, as examples of parameters. Following successful deployment, a chronic study may be used to assess effects of the resorbable foams on vaginal tissue after 30D (histology). Retrieved stents can be characterized to determine the extent of degradation and corollary effect on stent mechanical properties. Upon completion, there is development of a resorbable vaginal stent with self-fitting features that enables improved retention.

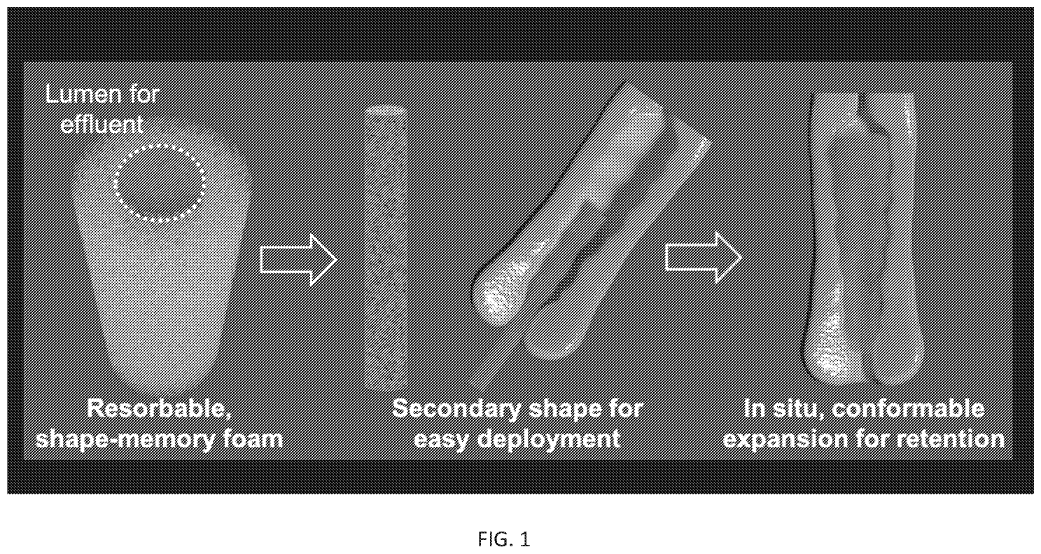

Specific embodiments utilize a biodegradable shape memory polymer (SMP) foam stent that may be comfortably inserted in a secondary, compressed shape. Upon insertion at a desired location, the increase in temperature from being inside the body (T˜36.5-37.8° C.) and hydration present at the location can initiate the expansion of the foam, thereby providing a conformable and personalized fit to the patient. In one specific example, upon insertion the increase in temperature of the vagina (T˜36.5-37.8° C.) (Zhang et al., 2013; Zhang et al., 2014) and hydration will initiate the expansion of the foam to provide a conformable and personalized fit to the patient and restore the lumen of the stent to allow egress of vaginal secretions, FIG. 1. However, current SMPs have not been explored for gynecologic products like vaginal stents and lack proper transition temperature and stiffness for vaginal stent applications. The present disclosure provides an iterative design to achieve target deployment temperature, mechanical properties, and resorption rate by leveraging the highly tunable chemical composition and foam architecture of this new SMP foam stent.

SMPs, such as polyurethanes (for aneurysm occlusion) and polyether-ether-ketone (PEEK, for orthopedic suture anchors), have been used in various medical devices in order to leverage their unique shape changing capacity, particularly shape expansion for securing in anatomy (Zhang et al., 2011; Nail et al., 2015; Woodard et al., 2017; Arora et al., 2015; Hsu et al., 2012). In some embodiments, the stent is comprised of or derived from a polymer selected from the group consisting of poly(vinyl alcohol) (PVA), poly-L-lactic acid (PLLA), polyglycolic acid (PGA), polyhydroxyethyl-methacrylate (PHEMA), poly(N-isopropylacrylamide) (PNIPAAm), [2-(methacryloyloxy)ethyl]dimethyl-(3-sulfopropyl)ammonium hydroxide (MEDSAH), 3-(acrylamidopropy)trimethylammonium chloride (AAPTAC), poly(ethylene glycol) diacrylate (PEG-DA), poly(tetrafluoroethylene) (PTFE), Poly(ε-caprolactone) (PCL), polycaprolactone diacrylate (PCLDA), polyvinylpyrrolidone (PVP), diacrylated poly(ethylene glycol) (PEG-DA), poly(2-acrylamide-2-methyl-propane sulfonic acid) (PAMPS), poly([2-(methacryloyloxy)ethyl]dimethyl(3-sulfopropyl)ammonium hydroxide) (PMEDSAH), polyurethane, polyether-ether-ketone, polydimethylsiloxane (PDMS), or any combination or copolymer thereof. In some embodiments, the stent can comprise a biopolymer, such as chitosan, hyaluronic acid, gelatin, alginate, methylcellulose, collagen, or any combination thereof. In some embodiments, polymer or copolymer properties can be adjusted to tune degradation rate and/or mechanical properties of the stent. In specific embodiments, the stent polymer or copolymer properties that can be adjusted include polymer molecular weight, dispersity, copolymer monomer ratio, inclusion of a crosslinking agent, and concentration of a crosslinking agent. The stent can be synthesized by a variety of methods, including but not limited to emulsion templating, salt leaching, gas-foaming, electro-spinning, and 3D-printing.

In some embodiments, the stent comprises a single network composition. A single network composition comprises a single type of polymeric component, and the stent comprises a homogeneous distribution of the single polymeric component. In some embodiments, the stent comprises a double network composition that comprises two polymeric components.

Embodiments of the present disclosure include methods that reduce the likelihood or severity of vaginal fibrosis or delay the onset of vaginal fibrosis. Methods of the disclosure also reduce or obviate the need for suturing of the vagina, such as following a medical procedure.

In some embodiments, the devices encompassed herein are of such a versatile design that they can be tailored for adult patients, for example those with post-radiation vaginal stenosis from gynecologic and/or colorectal cancer treatment.

### IV. Elution of Therapeutic Agents

In some embodiments, the device of the disclosure is manufactured such that the device comprises one or more therapeutic or other agents. The agent(s) may be eluted from device, as part of a coating on the device, a combination thereof, and so on. In specific embodiments, the agent(s) are useful as therapeutic for the tissue adjacent to the device, near the device, and so on, such as being wound-healing, diseased tissue-healing, and so forth. In specific embodiments, the device is configured such that being resorbable over a period of time allows for delivery of the agent or agents over the period of time. The agent(s) may be utilized for wound healing, scar prevention, prolapse prevention, fibrosis prevention, and/or long-term treatment for chronic or recurring medical conditions, such as bacterial vaginosis, yeast infection, graft-versus-host disease, fistula, lichen sclerosus, lichen planus, urinary conditions, including incontinence, to prevent leakage or prolapse, as a pessary, etc. The release of the agent from the device may be a modified-release, such as immediate-release, sustained-release, delayed-release, or a controlled-release whereby the rate of agent release is controlled.

In certain embodiments, the agent(s) is provided on one or more exterior surfaces of the device, or is incorporated within the device. In some embodiments, the agent can be mixed with a precursor polymer solution and become incorporated within the polymer matrix during fabrication with release occurring upon degradation of the polymer. In some embodiments, the agent can be incorporated within a device by placing the device in a solution of the agent and allowing the agent to adsorb to the surface of the device with release controlled by desorption rates. In some embodiments, the agent can be covalently grafted to functional groups on device polymeric chains. The covalent-attachment groups can be selected to be labile groups, e.g., ester or thio-β ester groups, that will become cleaved and release the agent in a physiological environment. In some embodiments, the agent can be incorporated into a hydrogel coating of the device with release controlled via swelling of the hydrogel.

In some embodiments, the time period in which an agent elutes from the device is substantially the same time period of use of the device, including as at least part of the device is resorbed by the body. In other cases, substantially all of the agent elutes from the device prior to partial or complete resorption by the body; in such cases, a sufficient amount of the agent is utilized in order to provide sufficient healing at the site of use.

In specific embodiments, the agent is a hormone, antibiotic, pain reliever, hemorrheologic, vasoconstrictive, anti-inflammatory, anti-fibrotic, wound-healing agent, radioprotective material, anti-fungal, contraceptive, or any combination thereof. In specific cases, the agent is a drug, such as AMD3100, tacrolimus, 2-octyl cyanoacrylate, Alevicyn, Artiss, Becaplermin, Betaine/polyhexanide, Cadexomer iodine. Collagenase, Dermabond, Eletone cream, Episalvan, Evicel, Fibrin sealant, Filsuvez, Hypochlorous acid topical, Lodosorb, NexoBrid, Oleogel-S10, Petrolatum & mineral oil topical, Prontosan, Proteolytic enzyme, Regranex gel, Santyl, TachoSil, Tisseel VH, Tropazone, progestin, etonogestrel, or a combination thereof. When the agent is a hormone, the hormone may be estrogen.

### V. Kits

Certain aspects of the present disclosure also concern kits containing devices of the disclosure or compositions to produce devices of the disclosure.

Kits may comprise one or more components, any of which may be individually packaged or placed in a container, such as a package, tube, bottle, vial, syringe, or other suitable container means. In some embodiments, the kit comprises the device or compositions to produce the devices, such as sealed in a package, including in a sterile environment, and the kit may optionally also include one or more bioactive agents that are comprised in a tube, bottle, vial, syringe, etc.

Individual bioactive agent components may be provided in a kit in concentrated amounts; in some embodiments, a component is provided individually in the same concentration as it would be in a solution with other components. Concentrations of components may be provided as 1×, 2×, 5×, 10×, or 20× or more, for example. Examples include at least hormone, antibiotic, pain reliever, hemorrheologic, vasoconstrictive, anti-inflammatory, anti-fibrotic, wound-healing agent, radioprotective material, anti-fungal, contraceptive, or any combination thereof. Specific agents include AMD3100, tacrolimus, 2-octyl cyanoacrylate, Alevicyn, Artiss, Becaplermin, Betaine/polyhexanide, Cadexomer iodine. Collagenase, Dermabond, Eletone cream, Episalvan, Evicel, Fibrin sealant, Filsuvez, Hypochlorous acid topical, Lodosorb, NexoBrid, Oleogel-S10, Petrolatum & mineral oil topical, Prontosan, Proteolytic enzyme, Regranex gel, Santyl, TachoSil, Tissecl VH, Tropazone, progestin, ctonogestrel, or a combination thereof.

The kit may be configured to allow for placement of the bioactive agent on the device at the point of care, or ahead of time of the point of care.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Pediatric Vaginal Stents

As noted elsewhere herein, there is a clinical need for pediatric vaginal stents.

One Embodiment of a Vaginal Stent: In specific embodiments the pediatric vaginal stent is a resorbable, self-fitting vaginal stent that can improve clinical outcomes and quality of life for pediatric and adolescent patients, such as following vaginal surgery. At least some design features may allow for include easy insertion, prevention of egress with Valsalva, and ultimately prevention of fibrosis and improved vaginal healing. In addition, the development of new resorbable vaginal stents eliminate the need for postoperative stent removal upon completion of use. In specific embodiments, the design utilizes a shape-memory polymer (SMP) foam that is biodegradable and can assume a secondary, compressed shape for ease of deployment. Upon insertion, the change in temperature and hydration will initiate the expansion of the foam to shape fit to the individual patient and restore the lumen of the stent to allow egress of vaginal secretions, FIG. 1. Uniquely, this circumvents the need for an external balloon to inflate the stent, a strategy common to current designs that causes challenges during clinical deployment. SMPs, such as polyurethanes (for aneurysm occlusion) and PEEK (for orthopedic suture anchors), have been used in various medical devices in order to leverage their unique shape changing capacity, particularly shape expansion for securing in anatomy. However, they have not been explored for gynecologic products like vaginal stents. The poly(e-caprolactone)-based SMP foams provide multiple methods to optimize the mechanical properties, shape recovery, and resorption kinetics to meet the multifaceted criteria of a pediatric vaginal stent. A resorbable vaginal stent would have direct extension as a therapeutic modality for the adult population as well. This versatile design can be readily tailored for post-radiation vaginal stenosis due to gynecologic and colorectal cancer treatment or other conditions including those in urogynecology, menopause, and dermatological gynecology, for example.

The vaginal stent designed for the pediatric population utilizes a completely different design than traditional stents by avoiding inflation of a silicone balloon to prevent egress. A resorbable SMP foam provides a number of potential benefits; however, there are no current SMPs used in gynecologic applications that can be utilized as a vaginal stent. To address this void, the present disclosure provides new SMP chemistry tailored for this application. Further, the disclosure encompasses the development of new testing apparati to screen candidate stents prior to animal studies.

Thus, the device of the present disclosure includes a resorbable, self-fitting vaginal stent that will expand upon deployment to provide a conformable fit and prevent fibrosis. To this end, the device may utilize, in at least some cases, a new SMP foam with target transition temperatures (to trigger expansion) and appropriate mechanical properties for gynecologic application. Deployment and retention of this new vaginal stent design may first be tested in a custom benchtop anatomical model, followed by in vivo assessment, such as in a rabbit model.

### Example 2

Fabricate Resorbable, Shape-Memory Foams that Provide an Improved, Conformable Fit

The present example concerns at least gynecologic products, such as vaginal stents, that utilize poly(e-caprolactone) (PCL)-based SMP foams. Grunlan et al. originally developed PCL-based self-fitting foams to treat irregular bone defects. Critically, their preliminary results indicate that the temperature to trigger expansion (transition temperature) can be reduced by tailoring PCL molecular architecture. One can established structure-property relationships to tune the transition temperature and foam architecture to achieve clinically-relevant deployment parameters (shape recovery, expansion time, mechanical properties). Then, an anatomy-specific vaginal benchtop model can be utilized that replicates the anatomy and forces in the vagina to screen candidate stents and ensure adequate deployment and retention prior to testing in vivo. Mechanical testing as a function of degradation can be used to ensure that the stent retains sufficient mechanical properties to maintain vaginal caliber over the target healing time, such as 4-6 weeks.

One can synthesize new SMP foams with transition temperatures that permit deployment at body temperature while maintaining desired mechanical properties and biodegradation kinetics. SMP foams based on PCL diacrylate (PCL-DA, Mn˜10 kg/mol) [“linear architecture” ] were previously prepared by the Grunlan Lab via solvent casting particulate leaching (Zhang et al., 2013; Zhang et al., 2014; Zhang et al., 2011). These foams exhibited a transition temperature (Ttrans) of ˜55° C., corresponding to the melt transition temperature. Thus, these foams could be shape fixed and subsequently expanded by heating to the Ttrans. The resulting foams (porosity>70%) were shown to have excellent shape fixity and recovery (Rf ˜100%, Rr ˜95%) and robust mechanical properties (E˜17 MPa, CS˜20 MPa). More recently, PCL-DA was combined with thermoplastic poly (L-lactic acid) (PLLA, Mn˜15 kg/mol) as a semi-interpenetrating network to prepare SMP foams with highly tunable biodegradation kinetics (up to 25× faster) and mechanical properties (modulus increased by up to 60%). Herein, new SMP foams can be specially formulated for the vaginal stent application, with a lower Ttrans for expansion at body temperature and reduced modulus to improve patient comfort. Briefly, a 4-arm PCL [“star architecture” ] can be synthesized via ring-opening polymerization of ε-caprolactone with a tetrol initiator, followed by end functionalization with acryloyl chloride to produce star PCL-tetracrylate (PCL-TA). Data shows that star PCL-TA SMP foams have a lower Ttrans range (˜40 to 50° C.) and a lower modulus more suitable for soft tissue. The PCL-TA foam properties can be further fine-tuned by incorporating polylactide (PLA) thermoplastics to prepare PCL-TA/PLA SMP foams with tunable biodegradation and mechanical properties. One can iterate aspects of star PCL-TA/PLA SMP foam properties (i.e. star-PCL-TA functionality (Raya-Rivera et al., 2014; Law et al., 2015; Patel et al., 2016; Zhang et al., 2013; Zhang et al., 2014; Zhang et al., 2011), arm molecular weight, ratio of PCL-TA:PLLA) to obtain target thermal transitions and shape memory behavior. In-house instrumentation can be used to determine properties of interest including Ttrans and percent crystallinity with differential scanning calorimetry (DSC, TA Instruments Q100) using established methodology (Zhang et al., 2013; Zhang et al., 2014; Zhang et al., 2011).

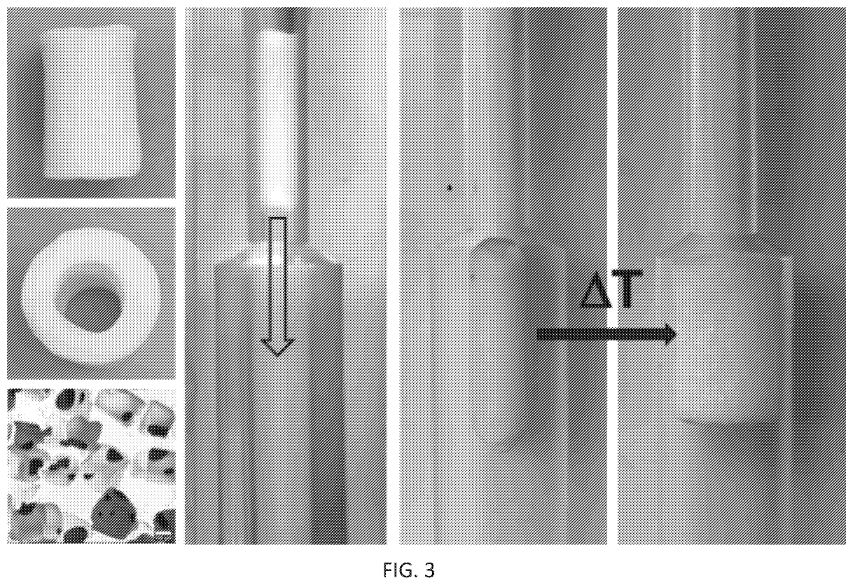

SMP Foam Fabrication: Following identification of SMP compositions that meet the targeted expansion temperature (Ttrans=35-40° C.), SMP foams can be fabricated following an adapted protocol from previously established methods (Nail et al., 2015; Woodard et al., 2017). Briefly, a slurry of NaCl particles (sieved to size) can be added to the mold, centrifuged, and dried in vacuo overnight to create a fused salt template with a defined lumen. The SMP macromere solution (with photoinitiator) can be added to the fused salt template and centrifuged to promote diffusion. After subsequent UV crosslinking (12 min, with 120o rotations every 4 min), the resulting crosslinked polymer can be air-dried overnight and the salt leached by soaking for four days in a 1:1 solution of EtOH:water with daily solution changes. Lastly, the foam can be allowed to air dry overnight and annealed with the central rod in place at 85° C. to create the final SMP foam, FIG. 3. Pore size and morphology can be characterized with scanning electron microscopy (SEM; image-J) and the percent porosity determined gravimetrically.

SMP Foam Testing: The effect of foam architecture on the Ttrans and percent crystallinity can be investigated using DSC. Initially, one can measure the properties of standard cylindrical specimens. The compressive modulus of cylindrical foams can be measured at storage temperature of 20° C. (<Ttrans) and at use temperature of 37° C. with an Instron testing frame using an environmental chamber. Shape fixity and shape recovery can be determined using strain-controlled cyclic-thermal mechanical compression tests over two cycles (DMA, TA Instruments RSA-III). The cylindrical foams can be subjected to the following sequence: (1) after equilibrating at 38° C. (Thigh) for 5 min, compress to a maximum strain at a rate of 50%/min, (2) hold at maximum strain for 5 min and then cool to 20° C. (Tlow) to fix the temporary shape, (3) remove the load and measure fixed strain and (4) reheat to 38° C. (Thigh) to recover the permanent shape with measurement of recovered strain. To start the second cycle, the specimen can be subsequently cooled to RT, reheated to 38° C. and then compressed to 50% of the height recovered during the first cycle. The time to recovery at 38° C. can be a criterion for clinical feasibility.

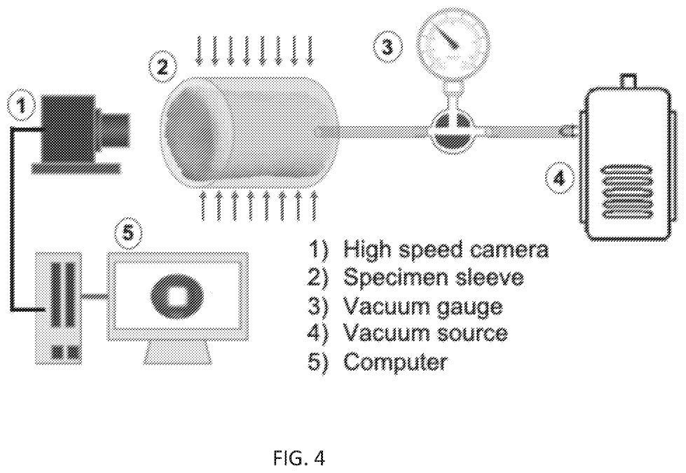

SMP Foam Stent Testing: The standard SMP characterization above provides important information to allow for iterative improvements in the foam design. However, the complexity of the foam stent geometry and the lack of direct measurements of the radial forces in the vagina make it difficult to use these measurements to directly select compositions that can maintain vaginal caliber, in certain embodiments. The only relevant literature reports of the physiological forces in the vagina were obtained using a variety of balloon-type pressure measurements (Arora et al., 2015; Hsu et al., 2012; Rosenbluth et al., 2010). These measurements are limited due to the averaging of the forces across the surface area of the balloon, and these measurements and do not capture the spatial heterogeneity of applied forces that may impact stent deformation, particularly buckling behavior. However, these clinically-relevant measurements do provide a useful starting point to assess the candidate foam compositions and iterate through the polymer design prior to in vivo testing. The percent shape recovery and deployment time of the SMP foam stents may first be tested in an acrylic deployment chamber held at 38° C. that replicates the vaginal diameter and length (FIG. 3). To characterize the mechanical properties of the foam stents, a custom test apparatus (FIG. 4) was designed to utilize the pressure measurements from human trials to determine if the candidate foams can maintain vaginal caliber after deployment (<10% deformation, retention of lumen). Briefly, one can place the SMP foam stents in a sleeve in a water bath at 38° C. and apply a negative pressure to the sleeve equal to the pressures measured in the vagina (7-45 kPa) (Arora et al., 2015). The negative pressure will generate a radial force from the sleeve to the outside of the foam. The deformation of the specimen geometry and foam compression will be captured with a high-speed camera and analyzed with ImageJ. Candidate compositions will be selected for similar testing coupled with in vitro real-time degradation analyses according to ASTM F1635 for 6 weeks using PBS solution with pH-4.5 (38° C.) to simulate the vaginal environment. In addition to changes in mechanical properties, gravimetric analysis of mass loss will also be used to determine the full degradation time frame. Findings from these customized testing setups can then be used to establish relative benchmarks to standardize SMP characterization for iterative testing, as needed. Foam porosity (70-90%) and pore size (100-400 mm) may be the targets for iterative design.

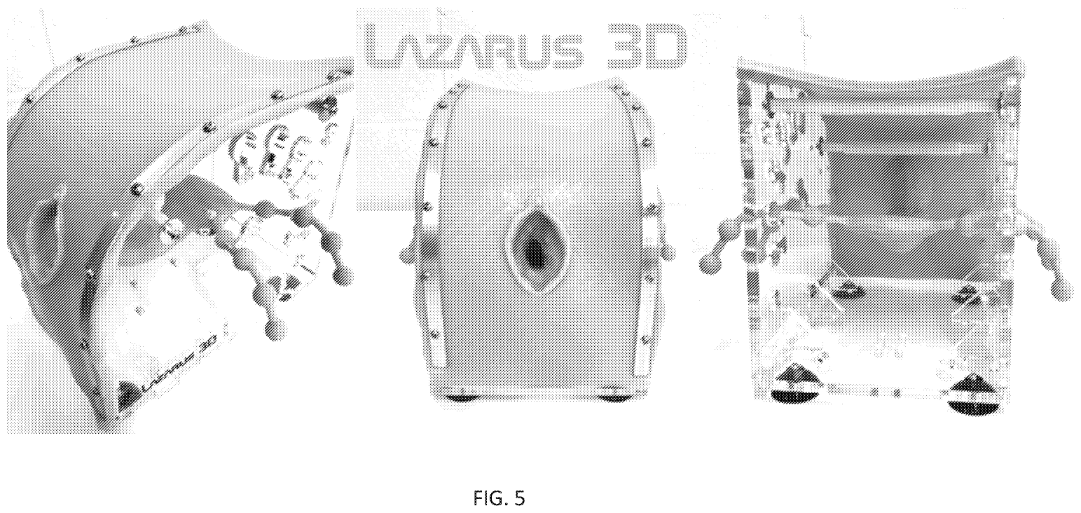

Confirmation of deployment and retention may be examined in a novel, vaginal benchtop model. Stent egress due to Valsalva forces is the largest clinical challenge of current vaginal stents, which frequently requires suturing of the vagina closed to prevent egress. To address this primary design criterion, candidate SMP foam composition identified as noted above is prepared and tested in a custom benchtop device that simulates the vaginal anatomy and Valsalva forces. First, a 3D printed pelvis comprising of the uterus, cervix and vagina from biocompatible silicone with soft labia/introital elements based on a representative adolescent pelvic MRI images was printed at Lazarus3D™, FIG. 5. Features of these pelvic structures include ability to repeatedly inject fluids into the model cavities to simulate blood or secretions and attachments via loop/peg locking system allowing different simulated uterine/vaginal tilts (anteverted, retroverted, axial) mimicking anatomic variations. This anatomic model is then encased in a pressurized acrylic casing that enables testing of stent deployment (time, expansion) and retention under simulated Valsalva force conditions (Emans et al., 2012; Lloyd et al., 2013; Raya-Rivera et al., 2014; Law et al., 2015; Patel et al., 2016; Zhang et al., 2013; Zhang et al., 2014; Zhang et al., 2011; Nail et al., 2015; Woodard et al., 2017; Arora et al., 2015; Hsu et al., 2012; Rosenbluth et al., 2010). Intra-vaginal pressure microsensors and transducers can line the vaginal canal capable of accurate, reliable, and continuous measurements of force applied from the stent to the vaginal walls. Notably, this model provides quantification of the radial forces that the stent applies against the vaginal walls at specific points of interest (introitus, mid-shaft of vaginal lumen, closest to vagino-cervical junction). One can conduct measurements during deployment, under resting conditions, with Valsalva pressures applied externally to the vagina, and during material resorption. This feature allows for an improved capture of dynamic information not available in current testing systems.

In specific embodiments, there are several gates for successful SMP foam composition: 1) transition temperature in the range of 35-40° C. with full shape recovery in <30 min; 2) sufficient foam modulus to withstand collapse of vaginal canal. The custom benchtop vaginal model can be used to confirm appropriate deployment (full expansion in <30 min) and retention under simulated Valsalva forces. The radial forces measured during stent deployment may not be used as a gate for stent composition identification, in certain cases; however, this data can aid in the analysis of in vivo studies and can be useful for device development. Finally, retention of mechanical properties for 4 weeks (<20% loss) and full degradation in 3-4 months can be used as a success criterion. This target degradation rate was selected to ensure that the device can maintain vaginal caliber such that vaginal walls do not appose each other during the healing process (1-12 weeks or any range therebetween).

### Example 3

### Assessment of Resorbable Foams in Rabbit Model to Confirm In Vivo Deployment and Retention

Rabbits are frequently used as a gynecologic model and can provide complementary information regarding deployment, retention, and tolerance of our vaginal stents (Abramov et al., 2007). The benchtop testing using the novel pressurized vaginal model described elsewhere herein allows quantification of stent retention against known Valsalva pressures and the pressure of the stent against the vaginal walls at specific points of interest. The animal studies can provide information not available with in vitro testing, including 1) vaginal stent retention with movement-induced conformational changes to vaginal axis; 2) the impact of the vaginal stent against adjacent organs such as bladder and rectum; 3) comfort level with stent in situ; 4) host response to vaginal stent over time. To this end, one can adapt the stent composition to sizes appropriate for testing in the rabbit model. Acute testing can assess the ease of deployment, shape fit with imaging, and retention in the vagina (see below). Following successful deployment, the effect of the resorbable foams on vaginal tissue is assessed using histology and qPCR or other scientific methods. Extent of degradation and corollary effect on the mechanical properties will also be conducted on explanted stents (see below).

Confirmation of deployment and retention in an acute rabbit model is performed. One can first conduct acute studies to confirm that the candidate formulation can be deployed in a live animal with appropriate fit and retention. One can utilize intravital imaging to ascertain mechanical stretch parameters of the vaginal canal with stent in situ, appropriate fit and animal comfort, and retention despite Valsalva forces while allowing for egress of fluids. Given lack of appropriate available literature, this pilot study sample size (n=6) was based on multiple manuscripts examining degradable stents in rabbit esophageal stricture model (Yang et al., 2019; Shang et al., 2020; Ahu et al., 2017). The esophagus and vaginal canal are both mucosally lined lumens with comparative histological, physiological, and wound healing properties (Shang et al., 2020). Briefly, four naïve rabbits are imaged using x-ray fluoroscopy with instillation of vaginal contrast to determine average rabbit vaginal dimensions. A brief scouting study can be completed with two naïve rabbits in each of two treatment groups: resorbable and a control stent. A control balloon-type stent can be fabricated from a medical-grade silicone material known to be biologically compatible and non-toxic to vaginal tissue. Both resorbable and control stents are imbued with barium sulfate. Animals are sedated for stent placement and can undergo vaginal imaging using x-ray fluoroscopy immediately after maximal vaginal stent expansion in vaginal canal [˜30 min]. Observations are made 1, 2, 4, 8, and 24 h post-stent placement to evaluate stent retention and behavioral responses to pain (i.e. excessive grooming, lethargy or restlessness, and lack of appetite). A modified Behavioral Pain Scale (BPS) may be used to evaluate animals with stent placement compared to controls. Egress of fluid may also be evaluated by tracking urine output that will act as a corollary to vaginal fluid egress. If the stent is retained and animal comfort and urine egress is assured, animals will progress through to studies referred to below. Any modifications necessary can be made after the scouting study, and the remaining animals can proceed through the above-outlined protocol.

Assessment of biocompatibility and biodegradation of vaginal stents in a chronic rabbit model is performed. Following successful deployment and retention for 24 h, one can proceed with evaluation of long-term stent retention, vaginal patency, and effects on vaginal tissue. Rabbits in which the stent is retained and animal comfort and urine egress is assured proceed directly to the following studies, provided animal health and comfort is maintained. Stents can remain in the vaginal cavity for a total of 30 days. Rabbits will be closely observed 24 h after stent placement and twice weekly thereafter for behavioral distress. During observations each rabbit will be rated using BPS scoring as described above. Additionally, animals will be evaluated daily for stent egress. Vaginal imaging will be performed prior to stent removal at 30 days to evaluate vaginal canal dimensions, volume, and patency. Vaginal patency will be confirmed according to the following definition of patency: % patency=device volume/max vaginal volume. At the completion of the study, the vagina will be explanted for histology and gene analysis. Tissue will be fixed, sectioned, and evaluated for vaginal histology using standard H&E staining with pathologist evaluation for evidence of tissue necrosis.

Changes in collagen content are evaluated using Picro Sirius Red staining. Tissue may also be evaluated using qPCR inflammation array. Retrieved stents may be characterized for extent of biodegradation and the corollary effect on mechanical properties as described elsewhere herein.

Success criteria may include successful deployment of the vaginal stent in less than 30 min, retention despite Valsalva both acutely and at 30 days, rabbit discomfort less than 4 on the BPS (Raillard et al., 2019), and maintenance of urine output. At the tissue level, success may be defined as minimal to no differences in tissue morphology, necrosis, and collagen content between groups as well as minimal to no tissue inflammation in the rabbits exposed to the resorbable stent compared to control rabbits.

Quantitative characterization of SMP properties can be expressed as mean±standard deviation. Statistical comparisons can be made using the Student's t test for paired data and analysis of variance (ANOVA) for multiple comparisons with Tukey post hoc analysis for parametric data. Computations are performed using Prism at the significance levels of p<0.05. As this is a sex-specific application, only female animals may be used for analysis.

### Example 4

### Resorbable, Shape Memory Stents to Improve Vaginal Wound Healing

The present example concerns a resorbable, shape memory foam used as a potential vaginal stent material. Polycaprolactone diacrylate (PCLDA) was fabricated into a foam cylinder with a central lumen and the subsequent shape memory characteristics were evaluated to determine the feasibility of use as a self-fitting vaginal stent.

Polymer foams were fabricated via solvent casting particulate leaching with a centralized rod to create a lumen. A fused salt scaffold was prepared using a 425 μm sieved salt slurry with 7.5 wt % DI water. The slurry was transferred to a 50 ml conical tube with a central glass rod in place to create a lumen, centrifuged at 3200 RCF for 15 minutes and was allowed to dry in-vacuo overnight. After drying, a ˜10 wt % PCLDA DCM macromer solution was prepared and a 10 wt % 2-hydroxy-4′-(2-hydroxyethoxy)-2-methylpropiophenone photoinitiator solution in N-Vinylpyrrolidone was added to the macromer solution at 15 vol %. Sufficient volume of the resulting solution was added to the fused salt scaffold to cover the template, centrifuged at 1500 RCF for 10 minutes, UV crosslinked for 12 minutes with 1200 rotations every 4 minutes, and allowed to air dry overnight. Salt was leached from the composite in a 1:1 EtOH:DI water solution with daily changes for 4 days. The resulting foam was then allowed to air dry overnight and subsequently annealed at 85° C. for 1 hour to create the final foam. Shape memory behavior was then evaluated by warming the foam at 60° C. for 3 minutes. The foam was removed from heat and radially compressed until cool to lock in the temporary shape (˜5 minutes). The expansion at 60° C. and temperature range at which shape recovery occurred was evaluated.

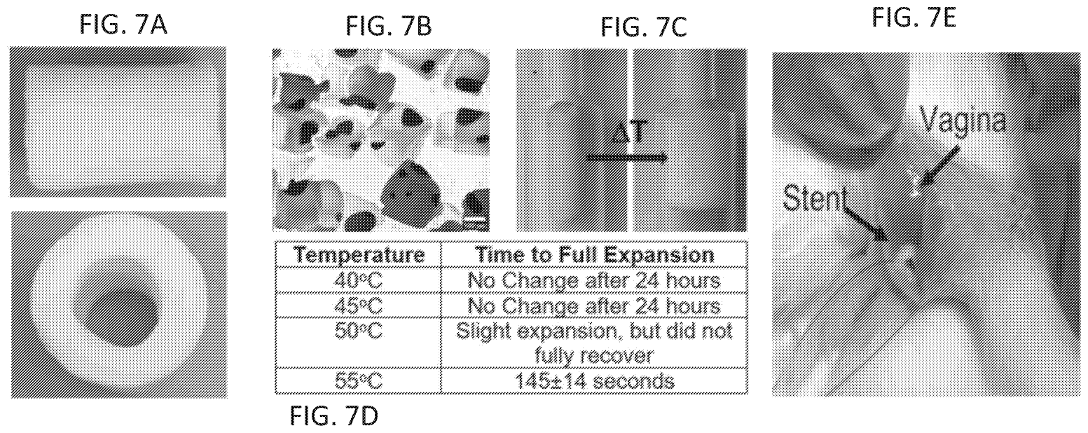

The fabrication method yielded a polymer tube (FIG. 7A) with a porous structure (FIG. 7B). The foam was able to be compressed after warming at 60° C. and subsequently expanded at temperatures above 55° C. (FIG. 7C). Insertion of the stent into the vagina of a rabbit model was used to demonstrate deployment feasibility (FIG. 7E) The deployment and self-fitting capabilities indicates the potential of shape memory polymer foams as novel vaginal stents. While shape recovery was determined to be 145±14 seconds at 55° C., limited or no recovery occurred at lower temperatures (FIG. 7D) indicating that relatively high temperatures are needed.

Overall, this evaluation of PCLDA foam fabrication and shape memory characteristics supported its use as a self-fitting vaginal stent that addresses many of the limitations of current vaginal stents. In certain embodiments, the polymer chemistry may be altered to reduce the transition temperature closer to body temperature to avoid potential pain and tissue damage. In addition, a novel platform may be utilized to evaluate the mechanical properties with regards to maintaining vaginal caliber within a simulated vaginal environment.

### Example 5

### Generation of Devices

[symbol]

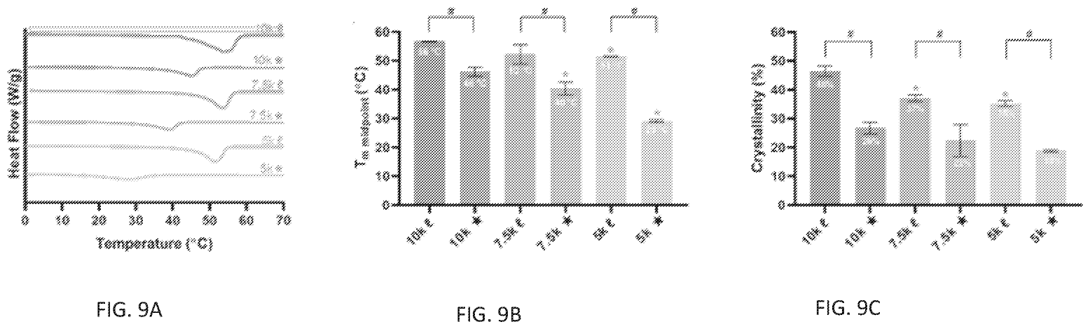

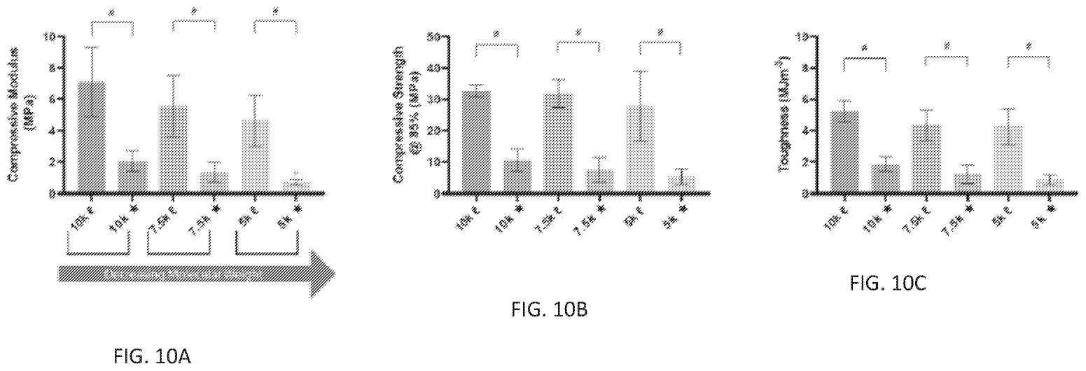

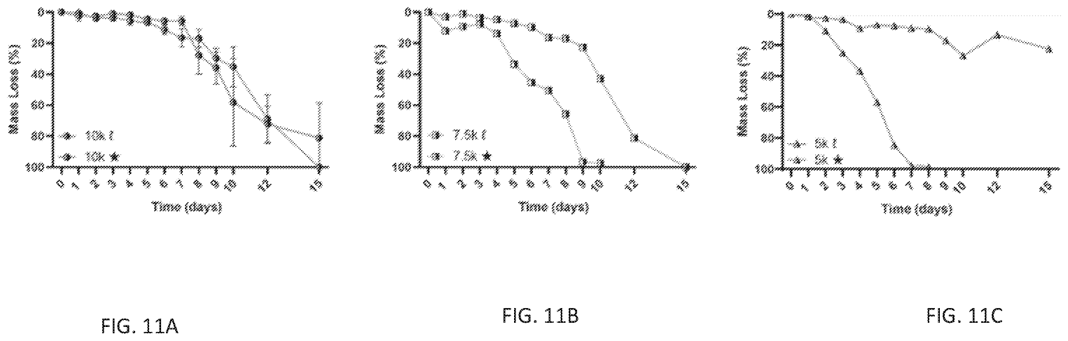

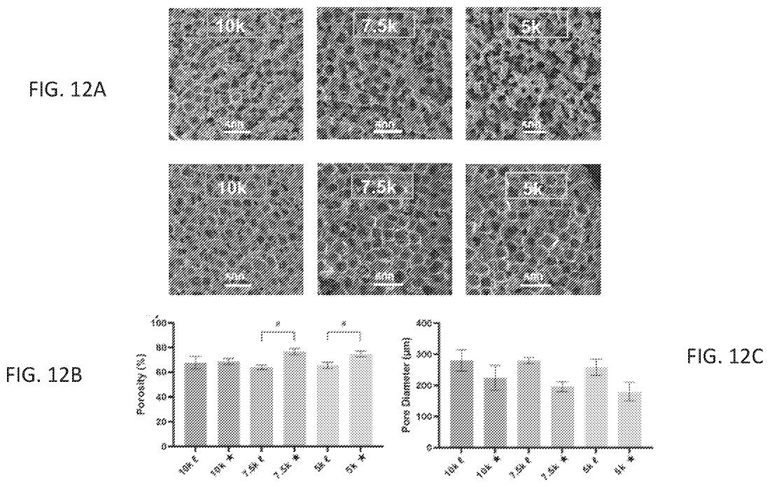

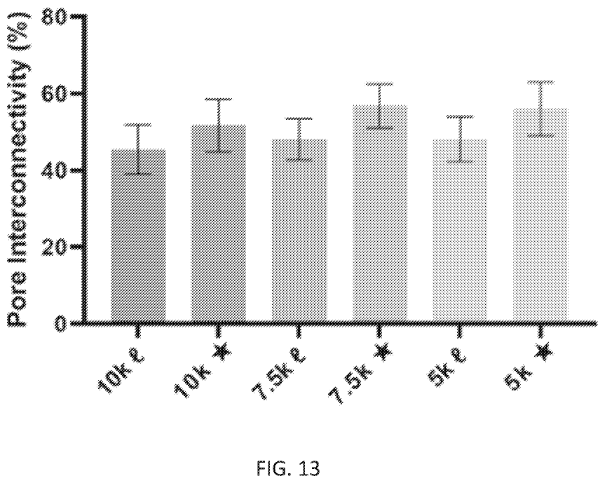

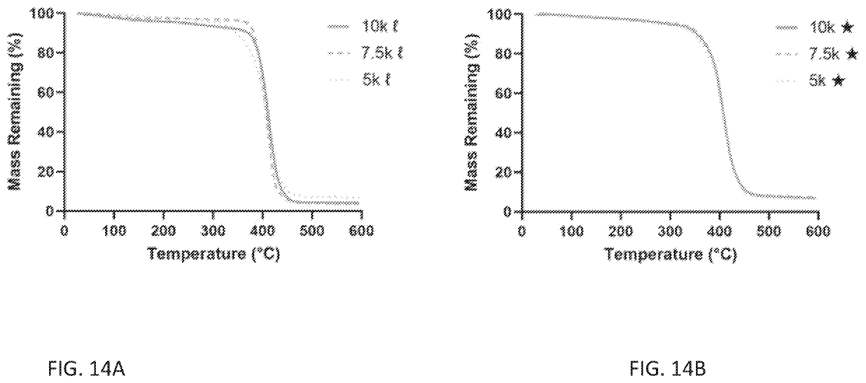

PCL-DA (“linear” architecture) and ★PCL-TA (“star” architecture) macromomers of varing molecular weight (Mₙ) were prepared. and used to fabricated scaffolds via solvent casting particulate leaching (SCPL) with a fused salt template. Scaffolds formed with ★PCL-TA exhibited reduced % PCL crystallinity and hence Tₘ values. A reduction in PCL-TA Mn decreased crystallinity and Tₘ as well. For instance, a scaffold prepared from PCL-TA (Mn˜5k g mol-1) exhibited a Tₘ of ˜29° C. and so would expand (“shape recovery”) upon insertion. Details that confirm this approach follow.
PCL (diol and tetrol) are prepared via ring-opening polymerization (ROP) of ε-caprolactone using an alcohol initiator (ethylene glycol or pentaerythritol, respectively) and stannous octoate as the catalyst. Molecular weight (Mn=10k, 7.5k, and 5k g mol-1) is modulated via ε-caprolactone to initiator ratio. Six macromer compositions were created: 10k [symbol] ( [symbol] PCL-DA, Mn˜10k g mol-1), 7.5k [symbol] ( [symbol] PCL-DA, Mn˜7.5k g mol-1), 5k [symbol] ( [symbol] PCL-DA, Mn˜5k g mol-1), 10k★ (★PCL-TA, Mn˜10k g mol-1), 7.5k★ (★PCL-TA, Mn˜7.5k g mol-1), and 5k★ (★PCL-TA, Mn˜5k g mol-1). FIG. 9 demonstrates the ability to create scaffolds with tunable Tin values (i.e., shape recovery temperatures) using ★PCL-TA macromers of varying Mn values. A Tm<body temperature is expected to provide self-expansion upon insertion (e.g., into the vaginal canal). FIG. 10 demonstrates the reduction in compressive mechanical properties as PCL crystallinity is reduced. A lower stiffness scaffold may provide greater comfort for use as a vaginal stent, etc.
FIG. 11 demonstrates the reduction that scaffolds with faster rates of degradation are produced when prepared from ★PCL-TA macromers (Mn˜7.5k and 5k g mol-1). This is expected to provide favorable rates of resorption in vivo, in some embodiments eliminating the need for removal (e.g., removal of a vaginal stent). FIGS. 12 and 13 demonstrate the reduction that porous scaffolds with pore interconnectivity can be prepared from [symbol] PCL-DA (“linear” architecture) and ★PCL-TA (“star” architecture) macromomers. Such macromers could be used in other fabrication processes (e.g., electrospinning, and emulsion templating).
FIG. 14 demonstrates that scaffolds prepared from [symbol] PCL-DA (“linear” architecture) and ★PCL-TA (“star” architecture) macromomers are effectively crosslinked, due to the lack of significant weight loss prior to catastrophic weight loss (˜400 deg C).

TABLE 1
Shape memory cycle data: cycle 2

| | R_{F CYCLE 2} | R_{R CYCLE 2} |
|---|---|---|
| 10K [symbol] | 101.92 ± 3.62 | 96.98 ± 1.90 |
| 10K ★ | 103.48 ± 4.16 | 96.42 ± 1.02 |
| 7.5K [symbol] | 102.28 ± 2.62 | 101.33 ± 3.46 |
| 7.5K ★ | 102.68 ± 5.52 | 101.18 ± 4.19 |
| 5K [symbol] | 95.25 ± 3.12 | 99.67 ± 1.64 |
| 5K ★ | 105.43 ± 2.28 | 91.37 ± 5.49 |

Table 1 demonstrates that scaffolds prepared from [symbol] PCL-DA (“linear” architecture) and ★PCL-TA (“star” architecture) macromomers exhibit excellent shape fixity (Rf) and shape recovery (Rr).

### Example 6

### Examples of Methods for Example 5

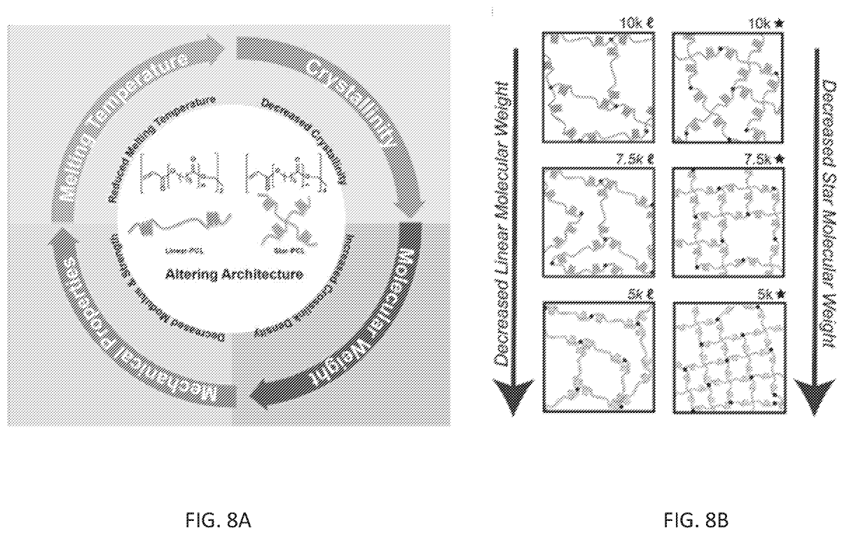

FIGS. 8A and 8B: SMP scaffolds were fabricated using solvent cast particulate leaching protocols to achieve porous scaffolds. NaCl was sieved and portioned into 20 mL scintillation vials (10.0 g, 425 um). DI water (7.5 wt %) was added in four additions followed by manual stirring after each addition. The wet salt was then compacted together using a glass stir rod and the vials were centrifuged (15 min, 3220×g). The vials were then air-dried for ˜1 hour and vacuum dried (RT, ON, 30 in. Hg). Macromer solutions were prepared by dissolving the desired macromer in DCM at 100 wt % (0.15 g total per mL DCM). Photoinitiator solution (10 wt % DMP in NVP) was then added at 15 vol %. To each salt template, 5 mL of the photoinitiator solution combined with the macromer solution were added and subsequently centrifuged (10 min, 1260×g). To crosslink the acrylated macromer solutions, vials were opened and exposed to UV light (UV-Transilluminator, 6 mW cm², 365 nm) for 6 min then left in the fume hood to air dry ON. To leach the salt from the scaffolds, vials were placed in a 1:1 ratio of water to ethanol for ˜5 days with daily solution changes. Once all the salt was dissolved out, scaffolds were left to dry in the fume hood ON and heat-treated the next day (85 C, 1 hr min, 30 in. Hg). The dried scaffolds (d×12 mm) were sliced into three specimens (t×2 mm) (Vibratome, Leica VT 1000 S) and were biopsy punched (Integra Miltex, 6 mm). The final specimen dimensions were d˜6 mm×t˜2 mm.

FIGS. 9A-9C: Thermal Properties. Differential Scanning Calorimetry (DSC; TA Instruments Q100) was used to determine Tₘ, Tₒₙₛₑₜ, and % Crystallinity of the PCL prior to scaffold fabrication and once fabricated into scaffolds (˜10 mg; N=3). Each specimen was sealed in hermetic pans and heated/cooled (10° C. min⁻¹) in two cycles. Reported values were determined using the second cycle to account for removal of thermal history in the polymers. The onset and midpoint temperatures were determined using TA Universal Analysis software where the maximum endothermic melt peak was analyzed to get the thermal properties as well as enthalpy of fusion. Percent crystallinity was calculated using equation (2)

$$\% {X}_{c} = \frac{Δ {H}_{m} - Δ {H}_{c}}{Δ {H}_{c}^{∘}}$$

Where ΔHₘ is the enthalpy of fusion calculated from the integral of the endothermic melt peak, ΔH_{c} is the enthalpy of crystallization from the exothermic cold crystallization peak and ΔH°_{c} is the theoretical value for 100% crystalline PCL (139.5 Jg⁻¹).

FIGS. 10A-10C: Compressive Properties. Scaffold specimens (d˜6 mm×t˜2 mm; N=5) underwent static compression testing (Instron 5944) at RT. Specimens were subjected to a constant strain (1.5 mm min¹) up to 85% strain. Due to their non-brittle nature, no specimen fractured. The average compressive modulus (E), strength (CS), and toughness were reported: E was determined from the initial linear region (<10% e). CS was determined from the stress at 85% strain. Toughness values were calculated from the area of the stress-strain curves up to 85% strain.

FIGS. 11A-11C: Accelerated Degradation. Specimens (d˜6×t˜2 mm; N=3 per timepoint) were submerged in 10 mL of 0.1 M NaOH in a 20 mL glass scintillation vials. Samples were placed in an incubator (37 C and 60 rpm). This study was conducted over 18 days with daily timepoints removed, rinsed with DI water, and dried in vacuo (RT; ON; 36 in. Hg). The final dried mass was compared to the initial mass to determine the mass loss (%).

FIGS. 12A-12C B5: Pore Size. SEM (Tescan Vega 3, Au—Pt sputter coating (˜4 nm), accelerating voltage˜10 kV) was used to analyze and visualize pore morphology. Scaffold images (N=3) were analyzed using image analysis software (ImageJ) and measurements were taken from pores along the diagonal midline to determine the average pore size.

Porosity. The percent porosity of scaffolds was determined by weighing and measuring the dimensions of films and porous scaffolds by using equation (1):

$$Porosity ( \% ) = ρ \frac{{ρ}_{solid film} - {ρ}_{porous scaffold}}{{ρ}_{solidfilm}} * 100$$

Where ρ_{porous scaffold} is the density of the final scaffold specimens and ρ_{solid film} is the density of the corresponding solid film.

FIG. 13: Pore Interconnectivity. Scaffolds (N=3; 6 mm diameter) were evaluated to determine their pore interconnectivity using a water wicking procedure. Scaffolds were submerged in 10 mL DI water and placed on a shaker plate (150 rpm, 24 hrs). Swollen scaffolds were removed and weighed on a petri dish. A folded Kimwipe was gently pressed onto the surface of the scaffolds for 1 min to wick away the interconnected water (Massₜₒₜₐₗ). The scaffold was then weighed again to get the Mass_{interconnected}

$$Interconnectivity ( \% ) = \frac{{Mass}_{total} - {Mass}_{interconnected}}{{Mass}_{total}} * 100$$

FIGS. 14A-14B: Thermal Degradation. TGA (TA Instruments Q50) of films (d˜4 mm×t˜1.5 mm, N=3) was performed under N2 from RT to 600° C. (heating rate=10 C min⁻¹) using platinum pans.

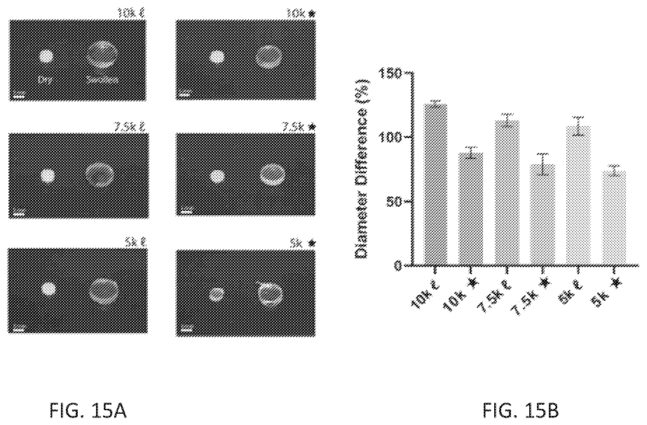

FIGS. 15A-15B: Crosslink Density. Crosslink density was qualitatively analyzed via a swelling test on solid films (d˜4 mm×t˜1.5 mm, N=3). Initial properties (diameter, thickness, and mass) were recorded then the films were submerged in 10 mL of DCM for 24 hours. Under a fume hood, films were removed while the swollen properties were taken. Pictures were taken of the initial and final film size to analyze the impact of crosslink density.

### Example 7

Self-Fitting Vaginal Stents from Biodegradable, Shape Memory Polymers

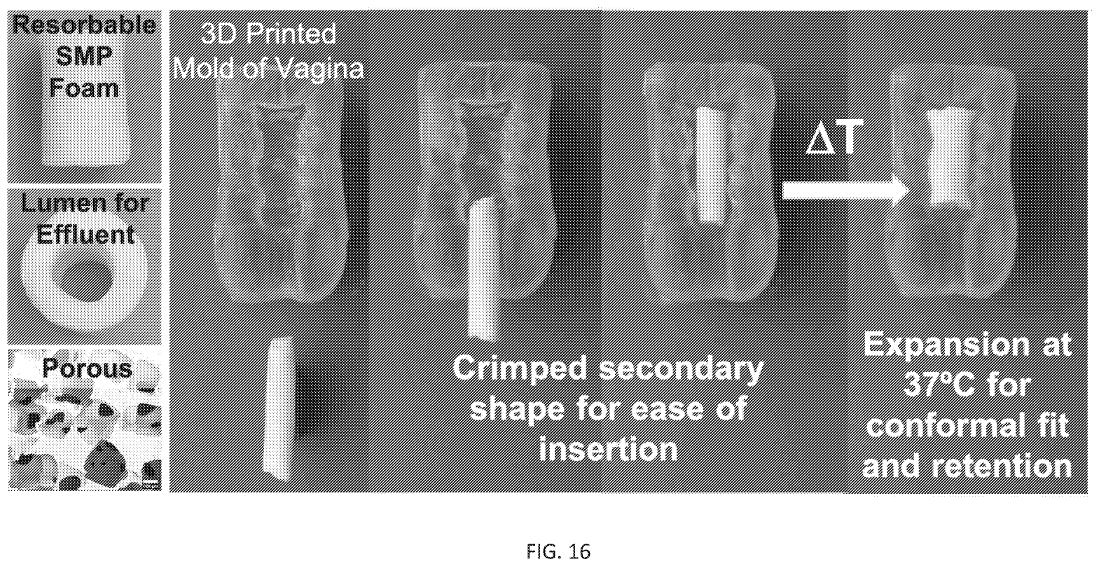

Approximately 50,000 female adolescent vaginal reconstructions are performed each year with 73% suffering from debilitating vaginal fibrosis post-surgery (Raya-Rivera AM, Esquiliano D, Fierro-Pastrana R, et al. The Lancet. 2014; 384(9940):329-336). Despite being one of the only ways to prevent complications and subsequent surgical revision, there is no commercially available vaginal stent for adolescents. Thus, physicians must create their own makeshift stents, using finger slots from gloves and gauze, cement coated in bone wax and placed in a condom, or plastic molding. There is a critical need for a new pediatric/adolescent-specific vaginal stent with appropriate sizing that can apply constant pressure to the boundaries of the neovagina to maintain vaginal caliber. It was considered that limiting tissue apposition during healing will ultimately prevent fibrosis. In addition, the development of single-use, resorbable stents offer additional advantages such as eliminating postoperative stent removal. To this end, the inventors developed a shape-memory polymer (SMP) foam stent that is biodegradable and can assume a secondary, compressed shape for ease of deployment. Upon insertion, the change in temperature and hydration initiates foam expansion to shape fit to the individual patient and restore the lumen of the stent to allow egress of vaginal secretions, FIG. 16. Herein is described the development of a new biodegradable SMP chemistry with target transition temperature and emulsion-templating approach to generate SMP foams with tunable pore structures.

Materials and Methods: Polycaprolactone diacrylate (linear-PCL-DA) and tetracrylate (star-PCL-TA) macromers were synthesized via ring-opening polymerization (ROP) (Pfau, M. A.; McKinzey, K. G.; Roth, A. A.; Graul, L. M.; Maitland, D. J.; Grunlan, M. A. J. Mater. Chem. B, 2021, 9, 3286-3837). Differential scanning calorimetry (DSC) was used to investigate the effect of structure and molecular weight (10, 7.5, 5 kDa) on the transition temperature (Tₘ) and % crystallinity. Foams were fabricated via an emulsion templating approach. Briefly, PCL-DA solution in toluene was emulsified with water (25:75) using the polyglycerol polyricinoleate 4125 (10% w/w) surfactant and 2.5% BAPO photoinitiator. The emulsion was photocrosslinked, allowed to air dry overnight, and subsequently annealed at 85° C. for 1 hour to create the final foam. SEM image analysis was used to characterize the pore structure. The effect of PCL solution concentration (10:90, 20:80, 30:70 PCL-DA:Toluene) and reactive diluent on pore architecture was investigated.
Results and Discussion: All formulations successfully formed stable emulsions; however, the lowest PCL-DA concentration (10:90) did not fully cure. This was attributed to the low concentration of PCL-DA preventing adequate crosslinking. Improved pore structures were observed with increasing PCL-DA concentration and NVP addition. Upon cure and drying, there is substantial densification of the continuous polymer phase. It was considered that at lower PCL-DA concentrations, this densification resulted in loss of the droplet-templated pore structure. At higher PCL-DA concentration, there was sufficient macromer concentration to retain the spherical droplet structure and the densification only resulted in pore opening at the thinnest portion of the impinging droplets with strut retention. Similarly, the NVP was expected to act as a reactive diluent in this formulation and promote additional crosslinking to preserve the emulsion-templated structure.
Linear-PCL-DA foams self-expanded at ˜50-55° C. (related to T_{m,PCL}˜55° C.), which was too high to allow for rapid expansion at physiological temperatures. New star-PCL-TA exhibited self-expansion at just 29-46° C., depending on macromer molecular weight.
Overall, this new SMP foam fabrication is useful in generating a self-fitting vaginal stent that addresses many of the limitations of current vaginal stents. New star-PCL-TA chemistry have successfully lowered the transition temperature to values that would allow for effective deployment at physiological temperatures. Current studies are focused on characterizing the mechanical properties, shape memory behavior, and resorption profile of these stents.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Abramov, Y.; Golden, B.; Sullivan, M.; Botros, S. M.; Miller, J. J. R.; Alshahrour, A.; Goldberg, R. P.; Sand, P. K., Histologic characterization of vaginal vs. abdominal surgical wound healing in a rabbit model. Wound repair and regeneration 2007, 15 (1), 80-86.
Arora, A. S.; Kruger, J. A.; Budgett, D. M.; Hayward, L. M.; Smalldridge, J.; Nielsen, P. F.; Kirton, R. S., Clinical evaluation of a high-fidelity wireless intravaginal pressure sensor. International urogynecology journal 2015, 26 (2), 243-249.
Emans, S.; Laufer, M.; Laufer, E., Goldstein's Pediatric and Adolescent Gynecology. Lippincott Williams & Wilkins Philadelphia, PA: 2012.
Hsu, Y.; Coleman, T. J.; Hitchcock, R. W.; Heintz, K.; Shaw, J. M.; Nygaard, I. E., Clinical evaluation of a wireless intra-vaginal pressure transducer. International urogynecology journal 2012, 23 (12), 1741-1747.
Law, E.; Kelvin, J. F.; Thom, B.; Riedel, E.; Tom, A.; Carter, J.; Alektiar, K. M.; Goodman, K. A., Prospective study of vaginal dilator use adherence and efficacy following radiotherapy. Radiotherapy and Oncology 2015, 116 (1), 149-155.
Lloyd, J. C.; Wiener, J. S.; Gargollo, P. C.; Inman, B. A.; Ross, S. S.; Routh, J. C., Contemporary epidemiological trends in complex congenital genitourinary anomalies. The Journal of urology 2013, 190 (4), 1590-1595.
Nail, L. N.; Zhang, D.; Reinhard, J. L.; Grunlan, M. A., Fabrication of a Bioactive, PCL-based. JoVE (Journal of Visualized Experiments) 2015, (104), e52981.
Patel, V.; Hakim, J.; Gomez-Lobo, V.; Oelschlager, A. A., Providers' experiences with vaginal dilator use for progressive perineal dilation (PPD). Journal of Pediatric and Adolescent Gynecology 2016, 29 (2), 159-160.
Raillard, M.; Detotto, C.; Grepper, S.; Beslac, O.; Fujioka-Kobayashi, M.: Schaller, B.; Saulacic, N., Anaesthetic and Perioperative Management of 14 Male New Zealand White Rabbits for Calvarial Bone Surgery. Animals 2019, 9 (11), 896.
Raya-Rivera, A. M.; Esquiliano, D.; Fierro-Pastrana, R.; López-Bayghen, E.; Valencia, P.; Ordorica-Flores, R.; Soker, S.; Yoo, J. J.; Atala, A., Tissue-engineered autologous vaginal organs in patients: a pilot cohort study. The Lancet 2014, 384 (9940), 329-336.
Rosenbluth, E. M.; Johnson, P. J.; Hitchcock, R. W.; Nygaard, I. E., Development and testing of a vaginal pressure sensor to measure intra-abdominal pressure in women. Neurourology and Urodynamics:Official Journal of the International Continence Society 2010, 29 (4), 532-535.
Shang, L.; Pei, Q. S.; Xu, D.; Liu, J. Y.; Liu, J., Novel detachable stents for the treatment of benign esophageal strictures. Experimental and Therapeutic Medicine 2020, 19 (1), 115-122.
Woodard, L. N.; Kmetz, K. T.; Roth, A. A.; Page, V. M.; Grunlan, M. A., Porous poly (ε-caprolactone)-poly (L-lactic acid) semi-interpenetrating networks as superior, defect-specific scaffolds with potential for cranial bone defect repair. Biomacromolecules 2017, 18 (12), 4075-4083.
Yang, K.; Cao, J.; Yuan, T.-W.; Zhu, Y.-Q.; Zhou, B.; Cheng, Y.-S., Silicone-covered biodegradable magnesium stent for treating benign esophageal stricture in a rabbit model. World journal of gastroenterology 2019, 25 (25), 3207.
Zhang, D.; Petersen, K. M.; Grunlan, M. A., Inorganic-organic shape memory polymer (SMP) foams with highly tunable properties. ACS applied materials & interfaces 2013, 5 (1), 186-191.
Zhang, D.; George, O. J.; Petersen, K. M.; Jimenez-Vergara, A. C.; Hahn, M. S.; Grunlan, M. A., A bioactive “self-fitting” shape memory polymer scaffold with potential to treat cranio-maxillo facial bone defects. Acta biomaterialia 2014, 10 (11), 4597-4605.
Zhang, D.; Giese, M. L.; Prukop, S. L.; Grunlan, M. A., Poly (F-caprolactone)-based shape memory polymers with variable polydimethylsiloxane soft segment lengths. Journal of Polymer Science Part A: Polymer Chemistry 2011, 49 (3), 754-761.
Zhu, Y.-Q.; Yang, K.; Edmonds, L.; Wei, L.-M.; Zheng, R.; Cheng, R.-Y.; Cui, W.-G.; Cheng, Y.-S., Silicone-covered biodegradable magnesium-stent insertion in the esophagus: a comparison with plastic stents. Therapeutic advances in gastroenterology 2017, 10 (1), 11-19.

1. A gynecological and/or urological device, comprising a self-fitting, resorbable shape memory foam.

2. The device of claim 1, wherein said foam is comprised of or derived from poly(vinyl alcohol) (PVA), poly-L-lactic acid (PLLA), polyglycolic acid (PGA), polyhydroxyethyl-methacrylate (PHEMA), poly(N-isopropylacrylamide) (PNIPAAm), [2-(methacryloyloxy)ethyl]dimethyl-(3-sulfopropyl)ammonium hydroxide (MEDSAH), 3-(acrylamidopropy)trimethylammonium chloride (AAPTAC), poly(ethylene glycol) diacrylate (PEG-DA), poly(tetrafluoroethylene) (PTFE), Poly(F-caprolactone) (PCL), polycaprolactone diacrylate (PCLDA), polyvinylpyrrolidone (PVP), diacrylated poly(ethylene glycol) (PEG-DA), poly(2-acrylamide-2-methyl-propane sulfonic acid) (PAMPS), poly([2-(methacryloyloxy)ethyl]dimethyl(3-sulfopropyl)ammonium hydroxide) (PMEDSAH), polyurethane, polyether-ether-ketone, polydimethylsiloxane (PDMS), or any combination or copolymer thereof.

3. The device of claim 1, wherein said device is configured for a gynecological or reproduction application.

4. The device of claim 1, wherein said device is configured for a urological application.

5. The device of claim 1, wherein the device is in the shape of a cylinder, a ring, a tube, the letter “T”, at least one letter “S”, a sphere, a wishbone, a cuboid, a trapezoid, or a combination thereof.

6. The device of claim 1, wherein the device comprises one or more loops, one or more bifurcations, one or more curves, or a combination thereof.

7. The device of claim 1, wherein the device is, or is part of, a patch, a graft, a contraceptive device, a stent, or a suppository.

8. The device of claim 1, wherein the size of the device is expandable upon placement in vivo.

9. The device of claim 1, wherein the device is in the shape of a cylinder, and the length of the cylinder prior to placement in vivo is in the range of about 0.5 inch to about 2 inches.

10. The device of claim 1, wherein the device is in the shape of a cylinder, and the radius of the lumen is about 0.1 inch to about 0.25 inch.

11. The device of claim 1, wherein the device is in the shape of a cylinder, and the width of the wall of the cylinder is about 0.05 inch to about 0.25 inch.

12. The device of claim 1, wherein the device is a vaginal stent.

13. The device of claim 1, wherein the size of the device is configured for a vagina of a pediatric, adolescent, or adult individual.

14. The device of claim 1, wherein the device is a vaginal ring or cervical ring.

15. The device of claim 1, further defined as comprising multiple devices of concentric cylindrical shapes.

16. The device of claim 1, wherein the device comprises an effective amount of one or more agents.

17. The device of claim 16, wherein the one or more agents elutes from the device, is a coating on the device, or both.

18. The device of claim 16, wherein the agent is for contraception, wound healing, scar prevention, or pathogen treatment.

19. The device of claim 16, wherein the agent is a hormone, antibiotic, pain reliever, hemorrheologic, vasoconstrictive, anti-inflammatory, anti-fibrotic, wound-healing agent, radioprotective material, anti-fungal, anti-viral, contraceptive, or any combination thereof.

20. The device of claim 16, wherein the agent is AMD3100, tacrolimus, 2-octyl cyanoacrylate, Alevicyn, Artiss, Becaplermin, Betaine/polyhexanide, Cadexomer iodine. Collagenase, Dermabond, Eletone cream, Episalvan, Evicel, Fibrin sealant, Filsuvez, Hypochlorous acid topical, Lodosorb, NexoBrid, Oleogel-S10, Petrolatum & mineral oil topical, Prontosan, Proteolytic enzyme, Regranex gel, Santyl, TachoSil, Tisseel VH, Tropazone, progestin, etonogestrel, or a combination thereof.

21. A method of treating a tissue in an individual, comprising the step of applying the device of claim 1 to tissue of the individual.

22. The method of claim 21, wherein the tissue is wounded tissue, diseased tissue, or where the individual has a medical condition associated with tissue in need of the device.

23. The method of claim 22, wherein the wound is in the urinary tract, vagina, fallopian tube, ovary, cervix, uterus, bladder, ureter, urethra, vas deferens, epididymis, testicle, rectum, or seminal vesicle.

24. The method of claim 22, wherein the diseased tissue is in the urinary tract, vagina, fallopian tube, ovary, cervix, uterus, bladder, ureter, urethra, vas deferens, epididymis, testicle, rectum, or seminal vesicle.

25. The method of claim 22, wherein the wound is from a medical procedure.

26. The method of claim 25, wherein the medical procedure is an obstetrical procedure.

27. The method of claim 22, wherein the wound or diseased tissue or medical condition comprises treatment after radiation and/or surgery, for fibrosis, post-operational treatment after gender reassignment, physical damage or injury, scarring for any reason, shortening and/or tightening of tissue from surgery and/or radiation, menopause, vaginal birth, post-operational treatment after vaginal birth, vaginoplasty, labiaplasty, prevention of pre-term birth, cervical incompetence, vaginal/uterine prolapse, bacterial vaginosis, yeast infection, lichen planus, lichen sclerosus, incontinence, pelvic inflammatory disease, ectopic pregnancy, genito-urinary symptoms associated with menopause, or a combination thereof.

28. The method of claim 21, wherein the device is in the shape of a ring or a cylinder and is applied to the vagina or cervix.

29. A method of treating a wound or diseased tissue or medical condition at a desired location in vivo of an individual, comprising the step of placing the device of claim 1 to the desired location, wherein following placement at the location, the device expands in size, thereby improving fitting at the desired location.

30. A method of manufacturing the device of claim 1, comprising the steps of:
1. (a) forming a biocompatible and resorbable three-dimensional device by one or more of the methods of: emulsion templating, salt leaching, gas-foaming, electro-spinning, and 3D-printing, wherein the three-dimensional device is comprised of or derived from a polymer selected from the group consisting of poly(vinyl alcohol) (PVA), poly-L-lactic acid (PLLA), polyglycolic acid (PGA), polyhydroxyethyl-methacrylate (PHEMA), poly(N-isopropylacrylamide) (PNIPAAm), [2-(methacryloyloxy)ethyl]dimethyl-(3-sulfopropyl)ammonium hydroxide (MEDSAH), 3-(acrylamidopropy)trimethylammonium chloride (AAPTAC), poly(ethylene glycol) diacrylate (PEG-DA), poly(tetrafluoroethylene) (PTFE), Poly(F-caprolactone) (PCL), polycaprolactone diacrylate (PCLDA), polyvinylpyrrolidone (PVP), diacrylated poly(ethylene glycol) (PEG-DA), poly(2-acrylamide-2-methyl-propane sulfonic acid) (PAMPS), poly([2-(methacryloyloxy)ethyl]dimethyl(3-sulfopropyl)ammonium hydroxide) (PMEDSAH), polyurethane, polyether-ether-ketone, polydimethylsiloxane (PDMS), and a combination thereof, and, optionally,
2. (b) either during or after production of said three-dimensional device, providing at least one therapeutic agent within and/or on at least one surface of the three-dimensional device.

31. The method of claim 30, wherein the therapeutic agent is a hormone, antibiotic, pain reliever, hemorrheologic, vasoconstrictive, anti-inflammatory, anti-fibrotic, wound-healing agent, radioprotective material, anti-viral, anti-fungal, contraceptive, spermicide, or any combination thereof.

32. The method of claim 30, wherein the device is placed at a desired location in vivo in an individual.

33. A method of maintaining an opening in tissue of an individual, comprising the step of placing the device of claim 1 in the tissue.

34. The method of claim 31, wherein the tissue is in the urinary tract, vagina, fallopian tube, ovary, cervix, uterus, bladder, ureter, urethra, vas deferens, epididymis, testicle, rectum, or seminal vesicle.

35. The method of claim 31, wherein the device comprises one or more therapeutic or contraceptive agents.

36. A stent, comprising self-fitting, resorbable shape memory foam.

37. The stent of claim 34, wherein said foam is comprised of or derived from polyesters including poly(F-caprolactone) (PCL), poly-L-lactic acid (PLLA), and polyglycolic acid (PGA), acrylated polyesters including polyether-based diacrylates including poly(ethylene glycol) diacrylate (PEG-DA), acrylated polyester including polycaprolactone diacrylate (PCLDA), polyurethane, silicones, poly(vinyl alcohol) (PVA), polyhydroxyethyl-methacrylate (PHEMA), poly(N-isopropylacrylamide) (PNIPAAm), [2-(methacryloyloxy)ethyl]dimethyl-(3-sulfopropyl)ammonium hydroxide (MEDSAH), 3-(acrylamidopropy)trimethylammonium chloride (AAPTAC), poly(tetrafluoroethylene) (PTFE), polyvinylpyrrolidone (PVP), poly(2-acrylamide-2-methyl-propane sulfonic acid) (PAMPS), poly([2-(methacryloyloxy)ethyl]dimethyl(3-sulfopropyl)ammonium hydroxide) (PMEDSAH), or any combination or copolymer thereof.

38. The stent of claim 36, wherein the stent is generally shaped cylindrically.
